(19) United States
(12) Patent Application Publication
(10) Pub. No.: US 2010/0068692 A1
(43) Pub. Date: Mar. 18, 2010

(54) ANTIFREEZE GLYCOPROTEIN ANALOGUES AND USES THEREOF

(75) Inventors: Robert N. Ben, Ottawa (CA); Roger Tam, Ottawa (CA); Pawel Czechura, Ottawa (CA)

Correspondence Address: RONALD I. EISENSTEIN;NIXON PEABODY LLP, 100 SUMMER STREET, BOSTON, MA, 02110, US

(73) Assignee: UNIVERSITY OF OTTAWA, Ottawa (CA)

(21) Appl. No.: 12/367,914

(22) Filed: Feb. 9, 2009

Related U.S. Application Data
(60) Provisional application No. 61/026,962, filed on Feb. 7, 2008.
(60) Provisional application No. 61/085,058, filed on Jul. 31, 2008.

(51) Int. Cl. C09K 3/18; C07K 14/00; A01N 1/02; A23L 3/34

(52) U.S. Cl. 435/1.3; 530/395; 252/70; 426/327

(57) ABSTRACT

An antifreeze glycoprotein comprising at least one C-linked saccharide according to the following general formula:

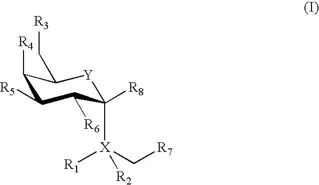

wherein X is nitrogen, sulfur, carbon or (CR¹R²)ₙS═O wherein n is 0, 1, 2 or 3, Y is carbon, nitrogen, sulfur or oxygen, R¹ is hydrogen, methyl, fluorine, a carbohydrate, or a hydroxy chain, R² is hydrogen, methyl, fluorine, a carbohydrate or a hydroxy chain, R³ to R⁶ independently represent an alkyl chain, a hydroxy group, a dimethyl sulfoxy group, a carbohydrate or an ether, R⁷ is ornithine, lysine, an alkyl chain, a pyridyl group or a heterocycle; and R⁸ is hydrogen, fluorine a carbohydrate or a hydroxy or methoxy group. Such antifreeze glycoprotein analogues are useful recrystallization inhibitors and may be used as a cryoprotectant for tissue preservation and transplantation, improving the texture of processed frozen food and frozen meats, frostbite protection, crop protection, and green alternatives for land vehicle antifreeze and aircraft de-icing.

### CROSS REFERENCE TO RELATED APPLICATION

This application claims benefit under 35 U.S.C. §119(e) of the U.S. provisional applications No. 61/026,962 filed on Feb. 7, 2008, and No. 61/085,058 filed on Jul. 31, 2008, the contents of which are incorporated herein by reference in their entirety.

### FIELD OF THE INVENTION

The present invention relates to synthetic antifreeze glycoprotein analogues that are useful for recrystallization-inhibition (RI) in aqueous substances and aqueous based systems, including cells, tissues, food, industrial fluids, and others.

### BACKGROUND OF THE INVENTION

Biological antifreezes are a diverse class of proteins found in fish, amphibians, plants and insects. These compounds have the ability to inhibit in vivo ice crystal growth and consequently, allow these organisms to survive sub-zero temperatures. This is a noncolligative phenomenon attributed only to biological antifreezes.

One class of biological antifreezes, the antifreeze glycoproteins (AFGPs) are isolated from Arctic and Antarctic teleost fish. These proteins range in molecular weight from 2.4 to 34 kDa and are composed of a tripeptide repeating unit (L-threonyl-L-alanyl-L-alanyl) where the L-threonine residue is glycosylated with the disaccharide β-D-galactosyl-(1,3)-α-D-N-acetylgalactosamine (FIG. 1) (Ananthanarayanan, V. S., Life Chemistry Reports 1989, 7, 1). The AFGPs of 2.4-2.7 kDa may have the threonine residue substituted with arginine and/or alanine substituted with proline (Raymond, J. A. L., Y.; DeVries, A. L., J. Exp. Zool. 1975, 193, 125; Hew, C. L. S., D.; Fletcher, G.; Shashikant, J. B., Can. J. Zool. 1981, 59, 2186; Morris, H. R. T., M. R.; Osuga, D. T.; Ahmed, A. I.; Chan, S. M.; Vandenheede, J. R.; Feeney, R. E., J. Biol. Chem. 1978, 253, 5155).

During the last decade, much effort has been devoted to understanding the mechanism by which AFGPs and other biological antifreezes function. One reason for this is the potential medical, commercial and industrial applications of these compounds, including tissue preservation and transplantation, improving the texture of processed frozen foods and frozen meats, frostbite protection, crop protection, and green alternatives for land vehicle antifreeze and aircraft de-icing. However, a better understanding of ice binding specificity and affinity must be achieved before such applications can be fully realized.

Key to understanding the mechanism of action and rationally designing biological antifreezes with enhanced stability and activity are detailed structure-activity relationship studies. The AFGPs are ideal candidates for such studies since they possess a well-conserved primary and secondary structure. However, complex glycans require lengthy and costly syntheses (Lowary, T. M., M.; Helmboldt, A.; Vasella, A.; Bock, K, J. Org. Chem. 1998, 63, 9657). Two reasons for this are the instability of the anomeric carbon-oxygen bond under various reaction conditions (Elofs son, M. S., L. A.; Kihlberg, J., Tetrahedron 1997, 53, 369) and the need to employ orthogonal protecting group strategies. Few strategies for the synthesis of AFGPs and related analogues have been described over the last decade. These syntheses have employed solution phase (Anisuzzaman, A. K. M. A., L.; Navia, J. L., Carbohydrate Res. 1998, 174, 265) or continuous flow solid phase techniques (Filira, F. B., L.; Scolaro, B.; Foffani, M. T.; Mammi, S.; Peggion, E.; Rocchi, R, Int. J. of Biol. Macromol. 1997, 12, 41; Meldal, M. J., K. J., J. Chem. Soc., Chem. Commun. 1990, 483) and involve direct glycosylation of the peptide backbone or a stepwise elongation of the peptide backbone using a glycoconjugate. More recently, a diphenylphosphoryl azide (DPPA) mediated polymerization of a glycosylated tripeptide has been developed (Tsuda, T. N., S.-I., Chem. Commun. 1996, 2779). To date, none of these AFGPs and AFGP analogues have been tested for antifreeze protein-specific activity.

The present inventors have discovered that certain antifreeze glycoprotein analogues are effective recrystallization-inhibitors and can be synthesized efficiently. These antifreeze glycoprotein analogues can be used in a variety of industrial and medical applications in which recrystallization-inhibition is desired.

### SUMMARY OF THE INVENTION

The inventors have accordingly sought to provide new antifreeze glycoprotein analogues useful as recrystallization inhibitors and/or cryopreservants.

As an aspect of the present invention, there is provided an antifreeze glycoprotein comprising at least one saccharide according to the following general formula:

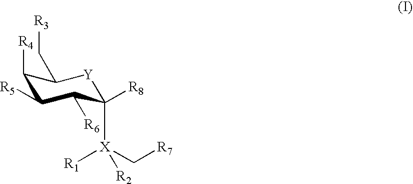

wherein

X is nitrogen, sulfur, carbon or (CR¹R²)ₙS═O wherein n is 0, 1, 2, 3,

Y is carbon, nitrogen, sulfur or oxygen,

R¹ is hydrogen, methyl, fluorine, a carbohydrate, or a hydroxy chain,

R² is hydrogen, methyl, fluorine, a carbohydrate, or a hydroxy chain,

R³ to R⁶ independently represent an alkyl chain, a hydroxy group a dimethyl sulfoxy group, a carbohydrate or an ether,

R₇ is ornithine, lysine, an alkyl chain, a pyridyl group or a heterocycle such as one of the following heterocyclic groups:

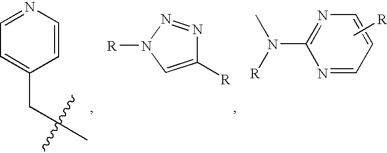

wherein R represents hydrogen, an alkyl chain or an aromatic group; and

R⁸ is hydrogen, fluorine, a carbohydrate or a hydroxy or methoxy group.

In certain embodiments, R⁷ is ornithine or lysine, and the ornithine or lysine is attached to the carbohydrate via an amide bond.

While X can be nitrogen, sulfur, carbon or (CR¹R²)ₙS═O as described above, in certain embodiments it may be preferred for X to be carbon. Similarly, it may in certain circumstances be preferred for Y to be oxygen, although it is possible that Y can be any of carbon, nitrogen, sulfur or oxygen as discussed above.

It is, in certain embodiments, preferred for R³ to R⁶ to each represent a hydroxy group. In other embodiments, R³, R⁴ and R⁶ may represent a hydroxy group while R⁵ defines a carbohydrate. The carbohydrate can, in select embodiments, comprise a saccharide such as galactose, glucose, fructose, L-fucose, lactose, melibiose or lactose(NAc) linked, for example, to the sugar moiety of formula (I) via an alpha- or beta-linkage.

In an alternative embodiment, R⁷ represents:

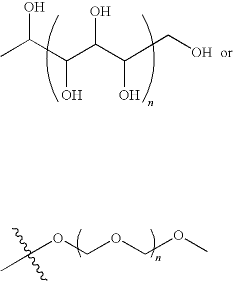

wherein n=1-100.

In certain embodiments, the carbohydrate is selected from galactose, glucose, fructose, L-fucose, lactose, melibiose and lactose(NAc). The carbohydrate may be the main saccharide unit of formula (I), or a substituent, e.g. in the case wherein R¹ to R⁶ or R⁸ represents or comprises a carbohydrate.

In alternative embodiments the main saccharide unit is represented by the following formulae (II) or (III):

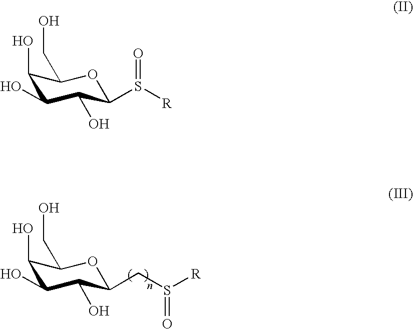

wherein R represents an alkyl chain or an aryl group. The aryl group may be a substituted or unsubstituted phenyl group, wherein the substituents include alkyl, ester, amide or carboxylic acid substituents. In such an embodiment, the main saccharide unit may alternatively be a glucose, fructose, L-fucose, lactose, melibiose, or lactose(NAc) unit.

In a further embodiment the main saccharide unit is a dimerized monosaccharide according to formulae (IV):

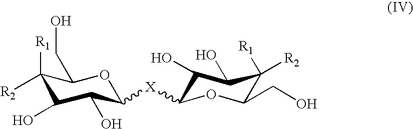

wherein R¹ and R₂ represent hydrogen, a halogen or a hydroxyl group, and X is CH₂, CF₂, oxygen or sulfur, and wherein the stereochemical nature of the dimer linkage is either alpha or beta.

In a further embodiment the main saccharide unit forms part of a glycoconjugate within the polypeptide chain. The polypeptide chain may comprise a repeating polypeptide unit. The repeating polypeptide unit can include several tripeptide repeats, typically 3 to 10 repeats, more particularly 3 to 5 repeats, and typically four repeats. As an example, in the case where R⁷ of formula (I) is ornithine, the repeating polypeptide unit would comprise (ornithine-aa¹-aa²)ₙ, wherein aa¹ and aa² each represent alanine or glycine, and n is 3 or 4. A similar embodiment would also be possible wherein R⁷ was lysine or serine. In certain non-limiting embodiments, the glycoconjugate may comprise four (ornithine-Gly-Gly) repeating units according to one of the following formulae (V), (VI), (VII) or (VIII):

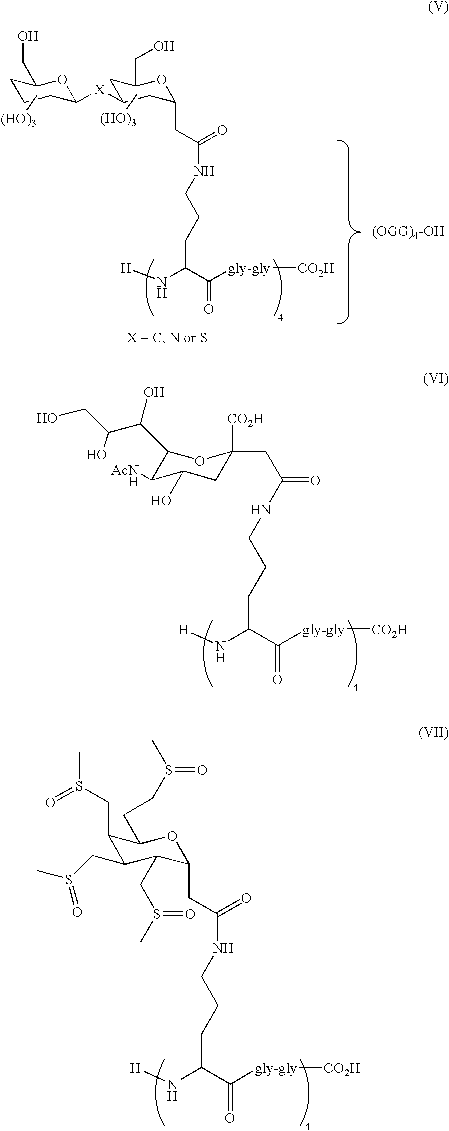

wherein formula (VIII) may be any variant of the above structures, i.e. a variant of Formula V, VI or VII, which comprises a peptide or amino acid replacement with a structure as follows:

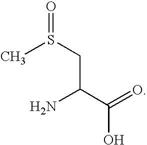

In a yet further embodiment the saccharide is attached to the polypeptide backbone through a flexible spacer.

Such antifreeze glycoprotein analogues are useful recrystallization inhibitors and may be used as a cryoprotectant for tissue preservation and transplantation, improving the texture of processed frozen food and frozen meats, frostbite protection, crop protection, and green alternatives for land vehicle antifreeze and aircraft de-icing.

Accordingly, there is also provided a method of inhibiting recrystallization, wherein the method comprises adding an antifreeze glycoprotein analogue as described herein to a material in need thereof in an amount sufficient to inhibit recrystallization. The method can therefore involve adding the antifreeze glycoprotein analogue to a material as a cryoprotectant for tissue preservation and/or transplantation, for improving the texture of processed frozen food, for frostbite protection, for crop protection, or is added to a composition for land vehicle antifreeze and aircraft de-icing.

Further provided is a saccharide unit for an antifreeze glycoprotein analog according to the following general formula:

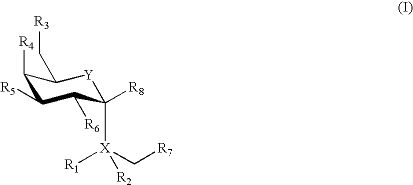

wherein:

X, Y, and R¹ to R⁸ are all as defined above, including all discussed variants. Such a saccharide unit is a useful building block for preparing the antifreeze glycoprotein analogues described herein.

Those skilled in the art will recognize, or be able to ascertain using no more than routine experimentation, numerous equivalents to the specific procedures and embodiments described herein. Such equivalents are considered to be within the scope of this invention and are covered by the following claims.

### BRIEF DESCRIPTION OF THE DRAWINGS

Further details of the invention will become apparent from the following description, taken in combination with the appended figures wherein:

FIG. 1 illustrates a typical prior art antifreeze glycoprotein (AFGP);

FIG. 2 illustrates a general synthetic strategy for preparation of structural mimics of AFGPs;

FIG. 3 is a graphic representation of the results of the recrystallization inhibition (RI) assay using compounds 10, 11, 12 and 13 (* all samples are dissolved in PBS; concentrations have been corrected so that total carbohydrate concentration is 0.021 mmole/L in all the samples);

FIG. 4 illustrates images of single crystals using a nanoliter osmometer; (A) in a glass of distilled water, and (B) in a 0.0033 mmole/L solution of 12 in ddH₂O;

FIG. 5 is a graphic representation of the RI activity of C-linked AFGP analogues 14 (D-Glucose analogue), 15 (D-mannose analogue), 16 (D-galactose analogue) and 17 (D-talose analogue);

FIG. 6 illustrates the partial molar compressibilities of monosaccharides in aqueous solution at 298 K (K₂° (s)×10⁴, cm³ mol⁻¹ bar⁻¹), with values given for dominant conformer in solution⁶;

FIG. 7 is a graphic representation of the RI activity of C-linked AFGP analogues 18a, 18b and 18c;

FIG. 8 shows the results of an analysis of C-Linked AFGP analogue 18a according to its ability to protect WRL-68 cells against cryoinjury during freezing and storage at −25° C.;

FIG. 9 is a graph of recrystallization-inhibition activity of various concentrations of D-galactose solutions in PBS;

FIG. 10 is a graph of RI activity of various concentrations of D-galactose in PBS solution. X-axis represents the log 10(Concentration) of D-galactose solution;

FIG. 11 is a graph of RI Activity of various monosaccharides (1-4) and disaccharides (5-9) at 0.022 M in PBS solution;

FIG. 12 is a graph of RI Activity of various monosaccharides (1-4) and disaccharides (5-9) at 0.022 M in PBS solution, plotted against their respective hydration numbers;

FIG. 13 is a graph of RI Activity of carbohydrates (1-9), plotted against their respective Hydration Index (hydration number/partial molar volume) (mol¹cm⁻³);

FIG. 14 illustrates C-allylated derivatives of galactose (10, 14), glucose (11, 15), mannose (12), and talose (13);

FIG. 15 is a graph of RI Activity of native O-linked monosaccharides (1-4) and their C-glycoside derivatives (10-15) at 0.022 M in PBS solution;

FIG. 16 is a graph of RI Activity of various concentrations of DMSO and 0.022 M solutions of compounds (1) and (10) in PBS solution;

FIG. 17 illustrates a proposed mechanism for inhibition of recrystallization; shaded red represents hydrated solute, QLL=Quasi-liquid layer;

FIG. 18 illustrates a retrosynthetic analysis of the building blocks of C-Linked AFGP analogues 4-7;

FIG. 19 is a graph of RI Activity of 5.5 μM solution of native AFGP-8 (1), C-linked AFGP analogues 3-7, 23, 24 and PBS control solution;

FIG. 20 illustrates Circular dichroism spectra and deconvolution data for native AFGP-8, and C-linked analogues 3-7, 23, 24, dissolved in doubly distilled water. Solution conformation populations were estimated using IBASIS 5;

FIG. 21 illustrates variable-temperature ¹H-NMR (500 MHz) spectra and temperature coefficients (ppb/° C.) of amide protons of truncated monomer model systems for 26-28, in 95:5 (H₂O:D₂O) with DSS as an internal standard;

FIG. 22 illustrates (A) model AFGP analogue tripeptides (n=0-3, compounds 23-26, respectively). Torsional angles are shown in χⁿ and ψ, and (B) a summary of statistical analysis of MD simulations showing % Hydrogen-bonding occupancy, and average C_{α}-C₁ distance (Å);

FIG. 23 illustrates (A) free energy profile for χ² and χ³ torsional angles in 25; global minima at (χ²=−52.8°, χ³=−57.6° for 25 (box outlined in white) and (χ³=−177.6°, χ⁴=177.6° for 26 (box outlined in grey) are indicated; (B) free energy profile of the χ¹ torsional angle for 25-28 in kcal mol⁻¹; and (C) free energy profile of the Ψₛ torsional angle for 25-28 in kcal mol⁻¹; and

FIG. 24 illustrates calculated lowest energy conformations for glycopeptide monomers 25-28.

### DETAILED DESCRIPTION OF THE INVENTION

C-linked and similar glycoprotein analogues represent an important class of glycoproteins, as they result in a more stable compound, and enable cost effective synthesis of antifreeze glycoproteins. As with naturally occurring antifreeze glycoproteins the C-linked analogues, as well as the S-linked, N-Linked and O-linked analogues described herein, consist of a polyamide backbone with a sugar(s) appended to it. The usual O-linked native species are less stable, difficult to synthesize, and are cytotoxic to many human cell lines. They are extracted from a variety of organisms at a significant cost.

Herein described are synthetic antifreeze glycoprotein analogues that are designed with particular attention to decreasing solvation. Such analogues are useful for recrystallization-inhibition (RI) in aqueous substances and aqueous based systems, including cells, tissues, food, industrial fluids, and others.

There are different strategies for decreasing solvation, and thus synthetic AFGPs can be designed based on desired application for the end molecule. Design consideration can thus include degree of RI activity required, cost of production, toxicity and biodegradability. In certain embodiments, the AFGP analogues would be used at concentrations that are comparable to those used for natural AFGPs (≧5.54×10⁻⁶ M or 9 mg/L).

As mentioned, the primary consideration in designing synthetic antifreeze glycoproteins is to ensure that they discourage good solvation. In other words, they are able to significantly disrupt the order of the water around the glycoprotein. This can be done by designing the carbohydrate moiety such that it decreases the compatibility with the three dimensional hydrogen bonded network of water. Sugars with low molar compressibility value in the range of −4 to −4.5 cm³ mol⁻¹bar⁻¹ would be most effective. Decreased solvation can be measured using a Hydrophobic index. Alternatively, effect on solvation can be measured using Hydration index.

In addition, decreased solvation can be further achieved by attaching the carbohydrate using a short side chain that keeps the carbohydrate close to the backbone. This may be as short as a 2 carbon spacer. In an additional embodiment, an amide group can be introduced into the carbohydrate-backbone linker that is no more than 3 atoms between the carbohydrate residue and polypeptide backbone. As an example, ornithine can be used to link the carbohydrate to the backbone. In a yet further embodiment, a sugar can be incorporated wherein the C4 OH group is axial, and the C2 O group is equatorial, e.g. by using D-Galactose as the sugar moiety. In an additional embodiment an analogue can be designed whereby the compatibility of the hexose is inversely related to RI.

In a yet further embodiment, flexibility of the polypeptide backbone can be achieved by incorporating glycines. As an additional embodiment, the polypeptide backbone may have two (2) or more tripeptide units, and more preferably four (4) or more tripeptide units.

The antifreeze glycoprotein analogues may incorporate synthetic or natural sugars resulting in poor solvation. Some examples of such sugars include:

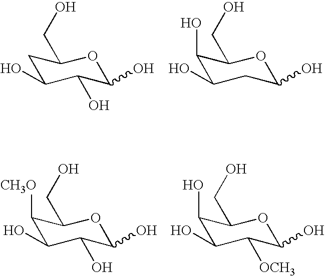

Additional embodiments will become evident from the following experiments, and are to be considered part of the inventive concepts described herein.

### DEFINITIONS

The term “alkyl” refers to a cyclic, branched, or straight chain alkyl group containing only carbon and hydrogen, and unless otherwise mentioned contains one to twelve carbon atoms. This term is further exemplified by groups such as methyl, ethyl, n-propyl, isobutyl, t-butyl, pentyl, pivalyl, heptyl, adamantyl, and cyclopentyl. Alkyl groups can either be unsubstituted or substituted with one or more substituents, e.g. halogen, alkyl, alkoxy, alkylthio, trifluoromethyl, acyloxy, hydroxy, mercapto, carboxy, aryloxy, aryloxy, aryl, arylalkyl, heteroaryl, amino, alkylamino, dialkylamino, morpholino, piperidino, pyrrolidin-1-yl, piperazin-1-yl, or other functionality. The term “alkyl” also encompasses the term “lower alkyl”, which refers to a cyclic, branched or straight chain monovalent alkyl radical of one to seven carbon atoms. This term is exemplified by such radicals as methyl, ethyl, n-propyl, i-propyl, n-butyl, t-butyl, i-butyl (or 2-methylpropyl), cyclopropylmethyl, i-amyl, n-amyl, hexyl and heptyl. Lower alkyl groups can also be unsubstituted or substituted, where a specific example of a substituted alkyl is 1,1-dimethyl heptyl.

“Hydroxyl” refers to —OH.

“Carboxyl” refers to the radical —COOH, and substituted carboxyl refers to —COR where R is alkyl, lower alkyl or a carboxylic acid or ester.

The term “aryl” or “Ar” refers to a monovalent unsaturated aromatic carbocyclic group having a single ring (e.g. phenyl) or multiple condensed rings (e.g. naphthyl or anthryl), which can optionally be unsubstituted or substituted with, e.g., halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, or other functionality.

The term “alkoxy” refers to a substituted or unsubstituted alkoxy, where an alkoxy has the structure —O—R, where R is substituted or unsubstituted alkyl. In an unsubstituted alkoxy, the R is an unsubstituted alkyl. The term “substituted alkoxy” refers to a group having the structure —O—R, where R is alkyl which is substituted with a non-interfering substituent. The term “arylalkoxy” refers to a group having the structure —O—R—Ar, where R is alkyl and Ar is an aromatic substituent. Arylalkoxys are a subset of substituted alkoxys. Examples of substituted alkoxy groups are: benzyloxy, naphthyloxy, and chlorobenzyloxy.

The term “aryloxy” refers to a group having the structure —O—Ar, where Ar is an aromatic group. A particular aryloxy group is phenoxy.

The term “heterocycle” refers to a monovalent saturated, unsaturated, or aromatic carbocyclic group having a single ring (e.g. morpholino, pyridyl or faryl) or multiple condensed rings (e.g. indolizinyl or benzo[b]thienyl) and having at least one heteroatom, defined as N, O, P, or S, within the ring, which can optionally be unsubstituted or substituted with, e.g. halogen, alkyl, alkoxy, alkylthio, trifluoromethyl, acyloxy, hydroxy, mercapto, carboxy, aryloxy, aryl, arylakyl, heteroaryl, amino, alkylamino, dialkylamino, morpholino, piperidino, pyrrolidin-1-yl, piperazin-1-yl, or other functionality.

“Arylalkyl” refers to the groups —R—Ar and —R—HetAr, where Ar is an aryl group. HetAr is a heteroaryl group, and R is a straight-chain or branched chain aliphatic group. Examples of arylaklyl groups include benzyl and furfuryl. Arylalkyl groups can optionally be unsubstituted or substituted with, e.g., halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, peperidino, pyrrolidin-1-yl, piperazin-1-yl, or other functionalities.

The term “halo” or “halide” refers to fluoro, bromo, chloro and iodo substituents.

The term “amino” refers to a chemical functionality —NR′R″ where R′ and R″ are independently hydrogen, alkyl, or aryl. The term “quaternary amine” refers to the positively charged group —N⁺R′R″R′″, where R′, R″ and R′″ are independently alkyl or aryl. A particular amino group is —NH₂.

All chemical compounds include both the (+) and (−) stereoisomers, as well as either the (+) or (−) stereoisomer.

Other chemistry terms herein are used according to conventional usage in the art, as exemplified by The McGraw-Hill Dictionary of Chemical Terms (1985) and The Condensed Chemical Dictionary (1981).

This invention is further illustrated by the following example which should not be construed as limiting. The contents of all references cited throughout this application, as well as the figures and table are incorporated herein by reference.

### EXAMPLES

### Experiment 1

### Synthesis of Structural Mimics of AFGPs

A general synthetic strategy was developed to prepare structural mimics of AFGPs (FIG. 2). The approach is centered on the synthesis of glycosylated tripeptide building blocks that are assembled using conventional solid phase synthesis. This approach differs from previous ones in that C-linked glycoconjugates are utilized. As a consequence, enhanced stability is obtained since C-linked glycoconjugates are not susceptible to acid/base or enzyme-mediated hydrolysis.

Synthesis of the glycosylated tripeptide building block is convergent in that saccharide and tripeptide components are covalently attached in a final step. Since early chemical and enzymatic modification of native AFGP (Komatsu, S. K. D., A. L.; Feeney, R. E., J. Biol. Chem. 1970, 245, 2909; Feeney, R. E. Y., Y., Adv. Protein Chem. 1978, 32, 191) demonstrated that the terminal galactose residue was crucial for activity, efforts have been focused on AFGP mimics that possess a truncated saccharide.

Scheme 1 outlines the synthesis of the saccharide component. C-Allylation of β-D-galactose pentaacetate with allyltrimethylsilane produced 3-[2,3,4,6-tetra-O-acetyl-D-galactopyranosyl]propene as a 80:20 mixture of α- and β-anomers.

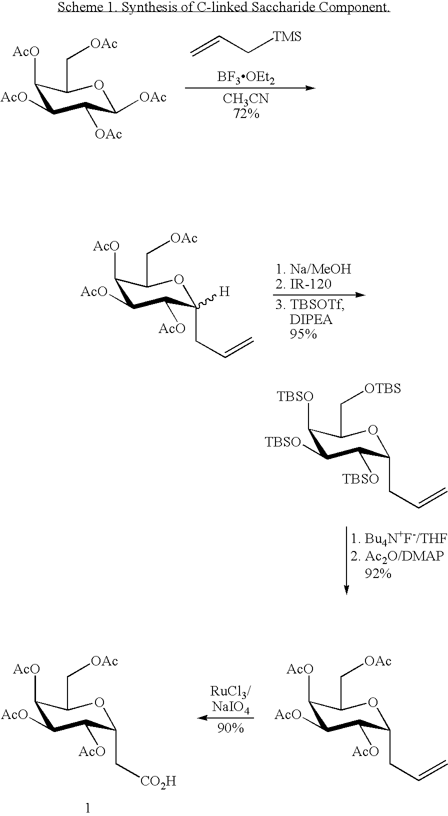

This mixture proved difficult to separate by column chromatography. To address this issue the acetate groups were replaced with less polar tert-butyldimethylsilyl groups. This was accomplished using standard literature procedures and as anticipated, the α- and β-anomers were easily separated by column chromatography. Desilylation and re-acetylation of the α-anomer was accomplished as a one-pot procedure with near quantitative yields and the resulting olefin was oxidized to furnish 1.

The tripeptide component was prepared as outlined in Scheme 2. Dipeptide 2 was synthesized by reacting commercially available Boc-glycine and glycine benzyl ester with 1,1′-carbonyldiimidazole (CDI) as a coupling agent.

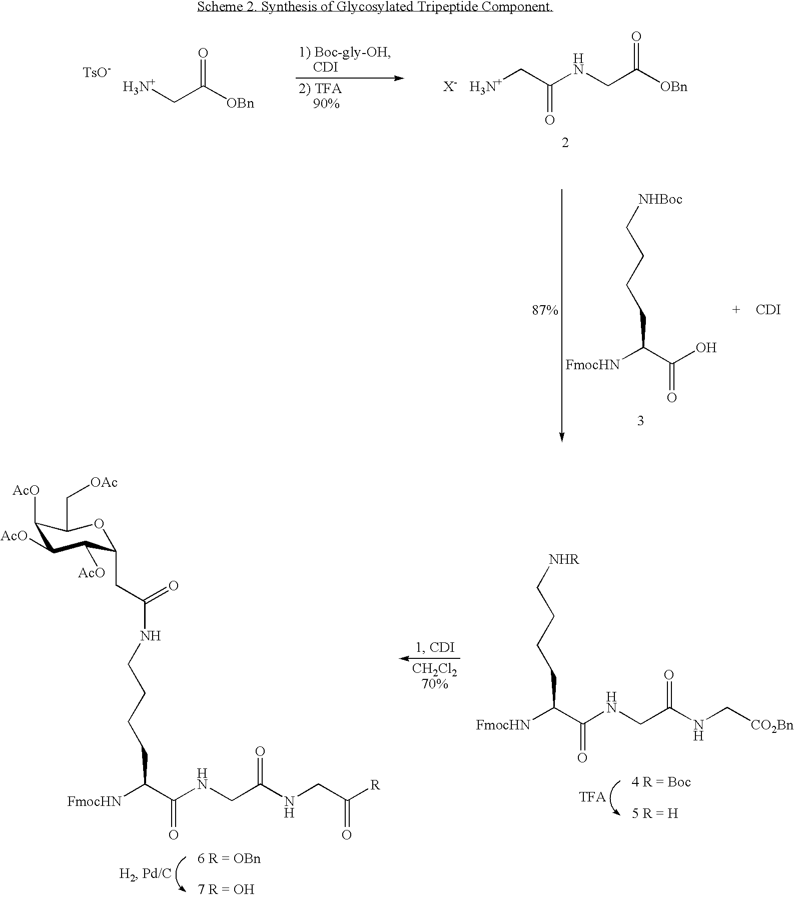

The dipeptide N-terminus was deprotected and then coupled to the commercially available lysine derivative 3 to give 4 in 87% yield. Removal of the tert-butylcarbamate and coupling of 1 to the 8-amino terminus produced 6. Upon hydrogenolysis, carboxylic acid 7, a structural analogue of the glycosylated L-arginine-L-alanine-L-alanine tripeptide unit found in lower molecular weight AFGP was produced.

The C-linked AFGP mimic was assembled from building block 7 by conventional solid phase synthesis using a Wang resin pre-loaded with Fmoc-glycine (Scheme 3). Successive couplings using O-(7-azabenzotriazol-1-yl)-N,N,N′,N′-tetramethyluronium hexafluourphophate (HATU) as coupling agent followed by cleavage from the resin resulted in glycoconjugate 8. After removal of the N-terminus protecting group and acetates, 9 was obtained in 55% yield.

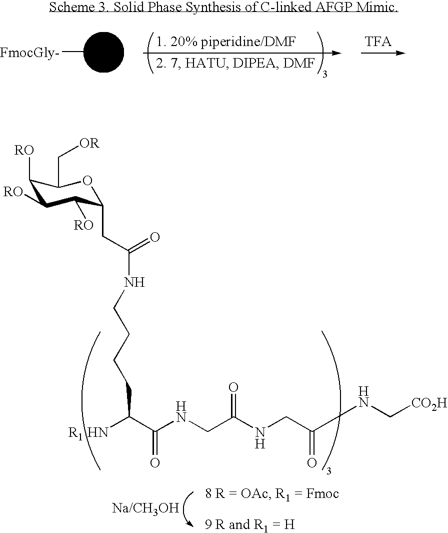

### AFGP's as Inhibitors of Recrystallization

FIG. 3 shows recrystallization inhibition (RI) assay results with the following examples of C-linked AFGP analogues:

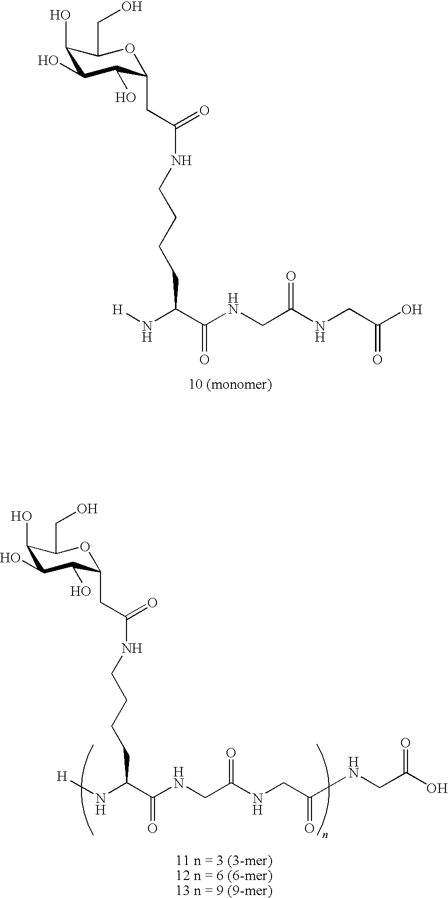

Compound 10 (monomer unit) as well as 11 (3-mer), 12 (6-mer) and 13 (9-mer) were assayed. All measurements were performed in triplicate. The Y-axis depicts the mean largest grain size (MLGS in mm²) of ice crystals measured directly from photographs of the sample, as described previously (Raymond, J. A. D., A. L., Proc. Natl. Acad. Sci. USA 1977, 74, 2589). To circumvent false positives due to additive carbohydrate concentrations amongst samples, the carbohydrate concentration of each sample was corrected to 0.21 mmol/L. For instance, compound 10 is 0.021 mmol/L with respect to both peptide and carbohydrate concentrations. Compound 11 is 0.007 mmole/L with respect to protein concentration but is 0.021 mmole/L with respect to carbohydrate concentration. Similarly, compound 12 is 0.021 mmole/L with respect to carbohydrate but 0.0033 mmole/L with respect to peptide. Both compound 13 and commercially available glycosylated Bovine Serum Albumin (BSA-conj., purchased as 20 mmole of (β-D-galactose/mmole of peptide) were treated in a similar fashion. The peptide control ((L-lysine-glycine-glycine)-6-glycine) is 0.021 mmole/L. PBS is used as the control since all samples are tested in a PBS solution.

Compounds 10 and 11 do not show any RI activity relative to PBS. However, compounds 12 and 13 possess RI activity. This is remarkable given the structural modifications of these compounds relative to AFGP 8. Furthermore, it is an interesting observation that 13 (n=9) appears to be slightly more active than 12 (n=6). This trend is consistent with the observation that lower molecular weight AFGPs (fractions 5-8) are less active than AFGP 1 (Eniade, A., Ben, R. N., Biomacromolecules 2001, 2, 557). Mindful of the relationship between glycoprotein length and antifreeze protein-specific activity, 12 and 13 were tested against an authentic sample of native AFGP-8, generously donated by AF Protein Inc. (data not shown in FIG. 3). AFGP-8 is the smallest of the AFGPs (n=4, 2.2 kDa) and is approximately 20× less active than AFGP 2-5. A direct comparison revealed that the analogues are weakly active (i.e. AFGP-8 is approximately ninety times more active than compound 13).

Non-specific RI effects (i.e. inhibition of ice growth) are common with colligatively acting substances such as inorganic salts, glycerol and oligosaccharides. In order to confirm that the activity of 12 was not a non-specific RI effect, several controls were also tested. The first was a peptide control ((L-Lysine-glycine-glycine)₆-glycine) composed of six tripeptide units analogous to 12, but with no sugars attached to the lysine side chains. As expected, this sample did not inhibit the growth of ice at concentrations equal to or even twice that of 12 and 13, highlighting the importance of the carbohydrate residues. This observation is consistent with earlier work demonstrating that the disaccharide residues in native AFGP are crucial to activity (Yeh, Y., Feeney, R. E., Chem. Rev. 1996, 96, 601). It was not possible to test the peptide control in the presence of uncoupled galactose because non-specific RI effects would be produced. Commercially available glycosylated BSA was also tested at a concentration of 0.21 mmole/L (relative to carbohydrate). As illustrated in FIG. 3, no RI activity was detected, suggesting that glycosylation is not the only important factor for RI activity. This result confirms that the antifreeze protein-specific activity observed with 12 and 13 is genuine and not the result of a non-specific RI effect.

Compounds 12 and 13 were tested for thermal hysteresis (TH) activity using the microcapilliary method. A very small TH gap was observed, unfortunately too small for an accurate measurement to be obtained. However, it was observed that ice growth did not occur at temperatures 0.06° C. below the melting point even after 15 hrs and when the temperature was lowered to 0.07° C. below the melting point, rapid crystal growth occurred and the entire solution froze within seconds. The fact that a crystal can be “held” for up to 15 hours at a temperature below its melting point is significant, considering that when solutions of other proteins (such as BSA, glycosylated BSA, salt, glycerol) are tested using this method, the solution freezes instantly at −0.01° C. below the melting point.

To further investigate the TH gap, compounds 12 and 13 were assayed using a nanoliter osmometer at concentrations identical to those used in the RI assay. These measurements confirm that 12 and 13 induce a small thermal hysteretic gap of 0.056° C. (30 mosmol). Looking at these results, 12 and 13 display similar activity in the TH assays, but have different activity in the RI assay. Considering this, it is important to note that the relationship between RI and TH activity is qualitative and not quantitative (Liu, S., Ben, R. N., C-Linked Galactosyl Serine AFGP Analogues as Potent Recrystallization Inhibitors. Org. Lett. 2005, 7, (12), 2385-2388).

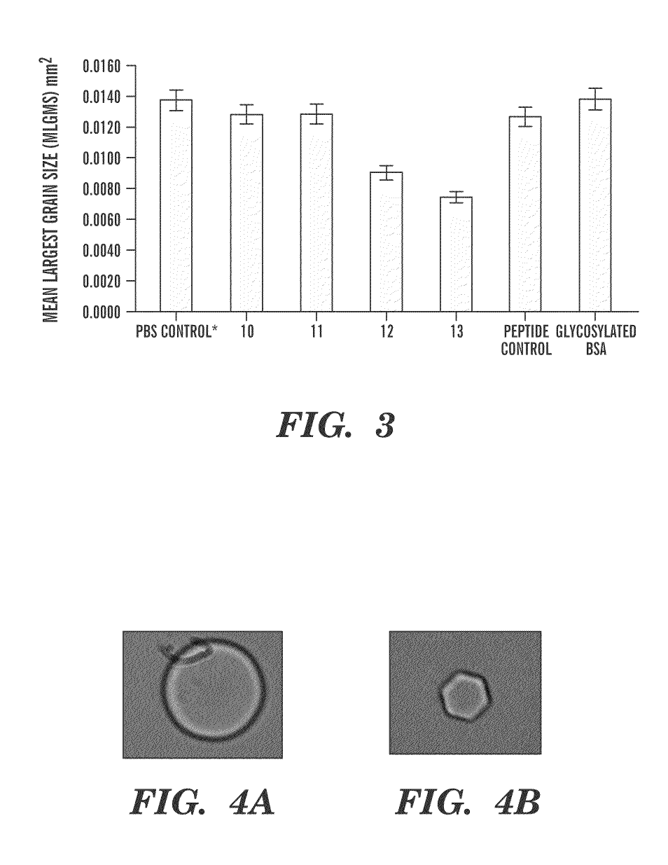

In addition to the observed TH gap, both 12 and 13 possess the ability to bind to ice as evidenced by unusual ice crystal morphology in the nanoliter osmometry assay. This “dynamic ice shaping” ability is a property unique to biological antifreezes and occurs when a biological antifreeze binds to the surface of an ice crystal. FIG. 4 (A) depicts a single ice crystal in the absence of biological antifreeze. Notice that the crystal is perfectly round and has no facets or edges. Identical images have been obtained when a single crystal is grown in the presence of BSA, glycosylated-BSA and sodium chloride. FIG. 4 (B) depicts a 0.0033 mmole/L solution (peptide concentration) of 13 in doubly distilled water. The single crystal is hexagonal, indicative of dynamic ice shaping and this hexagonal shape has been previously reported with weakly active mutants of the Type I AFP (Ben, R. N., Eniade, A. A., Hauer, L., Org. Lett. 1999, 1, 1759). Identical images were obtained for compound 13. These results verify that C-linked AFGP analogues 12 and 13 possess weak antifreeze protein-specific activity. Given the small thermal hysteretic gap and the fact that these compounds are smaller than AFGP 8, they may be ideally suited for the protection of cells during rewarming after freezing (Komatsu, S. T., DeVries, A. L., Feeney, R. E., J. Biol. Chem. 1970, 245, 2909).

### Stereochemistry of AGFP Analogues:

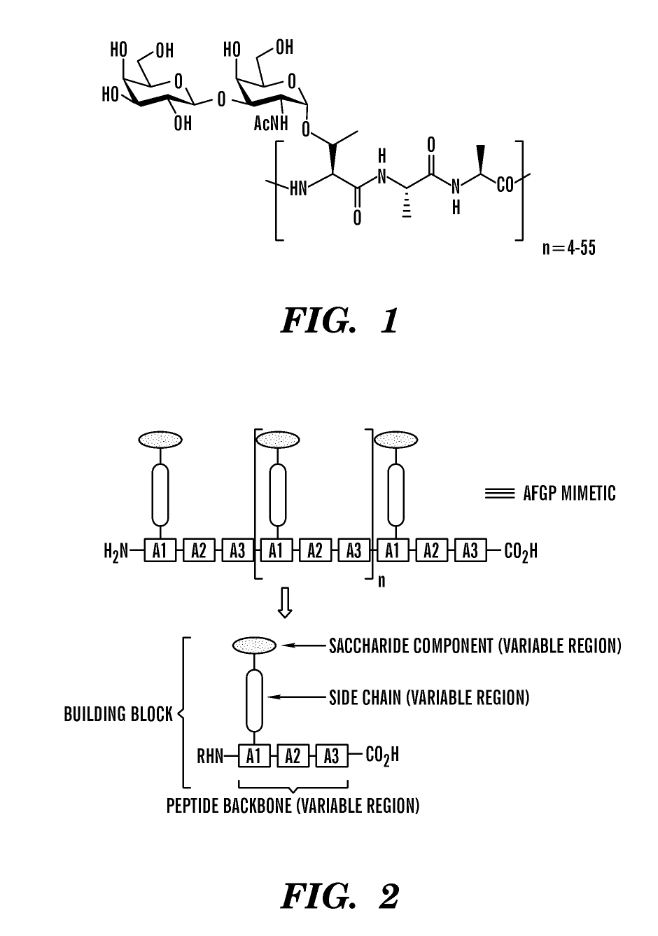

Four C-linked AFGP analogues, 14-17 (Scheme 4), were prepared in a convergent manner whereby the C-linked pyranose was coupled to an orthogonally protected L-ornithine residue and assembled into the desired glycopolymer using automated solid phase synthesis. Unlike previously reported analogues which possessed the L-lysine subunit in the repeating tripeptide (Ben, R. N., Eniade, A. A., Hauer, L., Org. Lett. 1999, 1, 1759; Eniade, A., Ben, R. N., Biomacromolecules 2001, 2, 557; Eniade, A., Purushotham, M., Ben, R. N., Wang, J. B., Horwath, K., Cell Biochem. Biophys. 2003, 38, 115), these analogues incorporated the L-ornithine residue. This was because the latter is a better structural mimic of the L-arginine residue, which is occasionally found in the lower molecular mass fractions of native AFGP (FIG. 1) (Yeh, Y., Feeney, R. E., Chem. Rev. 1996, 96, 601).

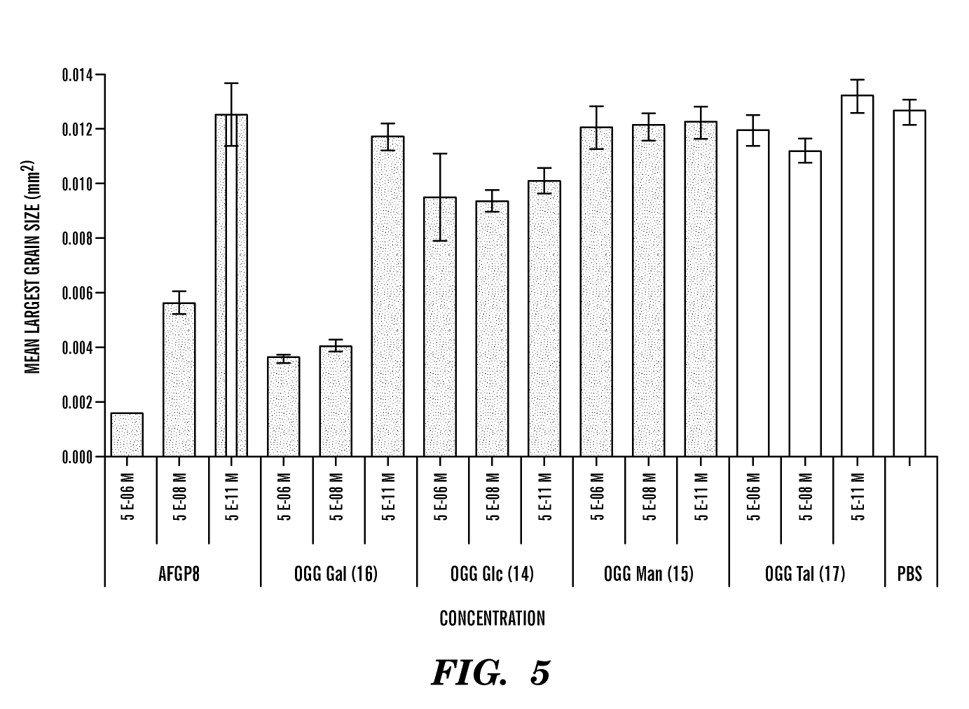

Analogues 14-17 were assessed for their ability to function as inhibitors of recrystallization (FIG. 5). All samples were compared to a solution of phosphate buffered saline (PBS) which was used as a negative control and a solution of AFGP 8 isolated from Gagus ogac (generously provided by AquaBounty Farms). Each solution was tested at three different concentrations in order to rule out any non-specific RI effects. The Y-axis in FIG. 5 represents mean largest grain size (MLGS) where small bars are representative of potent recrystallization-inhibition activity. The D-talose and D-mannose analogues (17 and 15) did not show any RI activity and had MLGS values consistent with PBS, the negative control. D-Glucose analogue 14 exhibited only very moderate RI activity while D-galactose analogue 16 was the most potent analogue with the largest MLGS value of 0.00354 mm² at 5.54×10⁻⁶ M.

The activity of this analogue is significantly greater than the L-Lysine AFGP analogue previously reported (Eniade, A., Purushotham, M., Ben, R. N., Wang, J. B., Horwath, K., Cell Biochem. Biophys. 2003, 38, 115). In addition, this analogue was composed of six or nine repeating tripeptide units while 16 has only four. Interestingly, analogue 16 is not as active as the C-Serine AFGP analogues synthesized previously (Liu, S., Ben, R. N., C-Linked Galactosyl Serine AFGP Analogues as Potent Recrystallization Inhibitors. Org. Lett. 2005, 7, (12), 2385-2388). Overall, analogue 16 is approximately two times less potent than native AFGP 8.

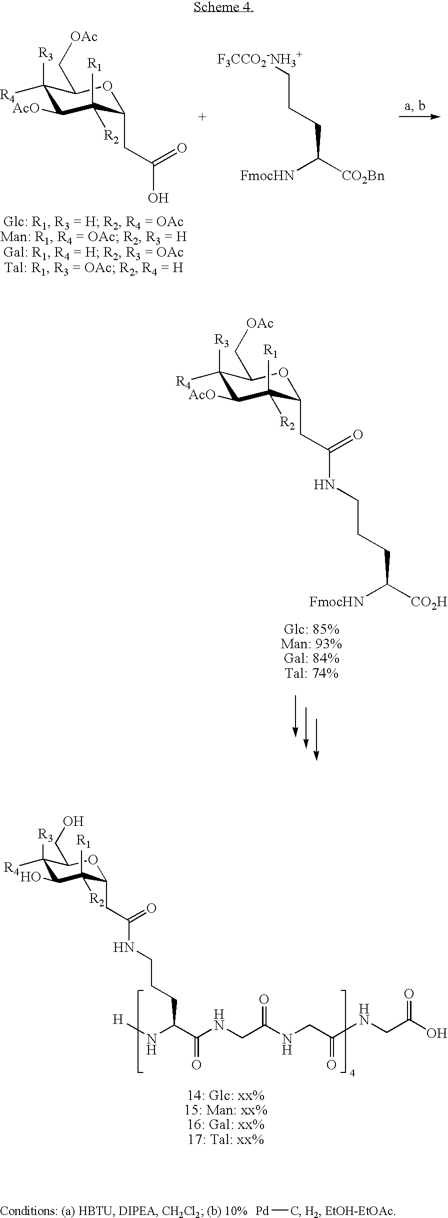

FIG. 5 represents an interesting result, where the stereochemical relationship of the pyranose ring is clearly important for recrystallation-inhibition (RI) activity. Specifically, the relative orientation between the OH groups at C2 and C4 are crucial. When the C4-OH group is axial and the C2-OH group is equatorial as in galactose-containing AFGP analogue 16, the largest amount of RI activity is observed.

This result compliments previous structure-function studies which demonstrated that the cis-3,4 hydroxyl group in the terminal D-galactose residue (Komatsu, S. T., DeVries, A. L., Feeney, R. E., J. Biol. Chem. 1970, 245, 2909), and the Gal-Ga1NAc dissacharide (Tachibana, Y., Fletcher, G. L., Fujitani, N., Tsuda, S., Monde, K., Nishimura, S. I., Angew. Chem. Int. Ed. 2004, 43, 856-862) are essential for thermal hysteresis activity. Recent studies by Corzona et al. (Corzana, F., Busto, J. H., Jimenez-Oses, G., de Luis, M. G., Asensio, J. L., Jimenez-Barbero, J., Peregrina, J. M., Avenoza, A., J. Am. Chem. Soc. 2007, 129 (30), pp 9458-9467), have suggested a correlation between the surrounding water shells of glycosylated serine and threonine residues with TH activity. However, identification of any of the key structural features of AFGP 8 for RI activity has yet to be addressed.

Given the stereochemical sensitivity of the carbohydrate moiety to RI activity and the Corzona precedent, an explanation for the increased RI activity of analogue 16 relative to analogues 17, 14 and 15 might lie in the hydration of these individual carbohydrate residues. An understanding of the structural characteristics essential to a potent inhibitor of recrystallization would greatly facilitate the rational design of novel cryoprotectants with custom-tailored activity. Such an approach is attractive as cell damage due to recrystallization is the single major cause of cell death during cryopreservation.

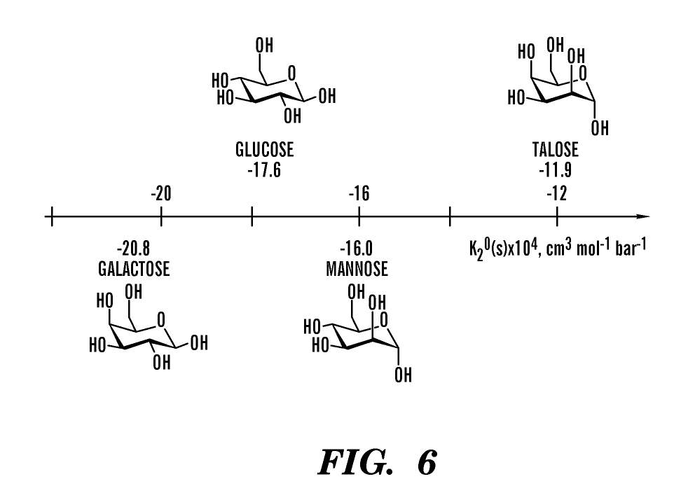

The hydration of carbohydrates has been studied extensively over the last five decades (Franks, F., Pure Appl. Chem. 1987, 59, 1189). Various hypotheses have been proposed to rationalize hydration characteristics of carbohydrates and protein and the subsequent influence of hydration on bulk water. The “hydration layer” is defined as water that encompasses the carbohydrate and/or protein and is often bound very tightly. In contrast, the region outside of this layer is very dynamic (consequently less ordered) and is referred to as “bulk water”. Hypotheses to rationalize hydration characteristics include: hydration number (Stokes, R. H., Robinson, R. A., J. Phys. Chem. 1966, 70, 16; Suggett, A., Ablett, S, Lillford, P. J., J. Solution Chem. 1976, 5, 17; Tait, M. J., Suggett, A., Franks, F., Abblett, S., Quikenden, P. A., J. Solution Chem. 1972, 1, 131; Uedaira, H., Uedaira, H., J. Solution Chem. 1985, 1985, (14), 7), anomeric effect (Kabayama, M. A., Patterson, D., Piche, L., Can. J. Chem. 1958, 36, 557), ratio of axial versus equatorial hydroxyl groups (Franks, F., Cryobiology 1983, 20, 335; Suggett, A., J. Solution Chem. 1976, 5, 33), hydrophobic index (Miyajima, K., Machida, K., Nagagaki, M., Bull. Chem. Soc. Jpn. 1985, 58, 2595), hydrophilic volume (Walkinsaw, M. D., J. Chem. Soc., Perkin Trans. 2 1987, 1903) and compatibility with bulk water based upon the position of the next-nearest-neighbour hydroxyl groups (Danford, M. D., J. Am. Chem. Soc. 1962, 84, 3965; Warner, D. T., Nature 1962, 196, 1055). Galema et al. have studied the hydration of various monosaccharides using molecular dynamics simulations, kinetic experiments, as well as density and ultrasound measurements (Galema, S. A., Hoiland, H., part 3. Density and Ultrasound Measurements. J. Phys. Chem. 1991, 95, 5321-5326; Galema, S. A., Howard, E., Engberts, J. B. F. N., Grigera, J. R., Carbohydr. Res. 1994, 264, 215-225). Consequently, the partial molar volumes and isentropic partial molar compressibilities of many commercially available hexoses have been determined. In general, these values relate to the volume of space occupied by a molecule upon solvation by water. This data reveals that the “compatibility” of a carbohydrate with the three-dimensional hydrogen-bonded network of water is governed by carbohydrate stereochemistry. Monosaccharide pyranoses with variable hydroxyl stereochemistry at C2 and C4 showed a relatively wide range of partial molar compressibilities. In other words, the ‘fit’ of monosaccharide pyranoses into the three-dimensional hydrogen bonded network is highly variable and dependant upon the C2 and C4 relative stereochemistry. The molar compressibilities of D-talose, D-mannose, D-glucose and D-galactose are shown in FIG. 6 where hexoses with low molar compressibilitity values exhibit the poorest fit with the three-dimensional hydrogen-bonded network of water (Galema, S. A., Hoiland, H., part 3. Density and Ultrasound Measurements. J. Phys. Chem. 1991, 95, 5321-5326).

A comparison of FIG. 5 to FIG. 6 reveals a strong correlation between recrystallization inhibition activity and molar compressibility of the monosaccharide hexoses. The data in FIG. 5 indicates that the compatibility of a hexose is inversely proportional to recrystallization-inhibition activity. When a molecule ‘fits’ well into the three-dimensional hydrogen-bonded network of water, less energy is required for solvation as minimal re-organization of the bulk water will be necessary. When a pyranose ‘fits’ well into the three-dimensional hydrogen-bonded network of water less re-organization of the bulk water is required and hence, the energy requirement for solvation is lower. The talose-containing AFGP analogue 17 exhibits no RI activity (FIG. 5). Based upon the fact that D-talose would have the best fit into the three-dimensional hydrogen-bonded network of water and thus demand the least re-organization of bulk water it should require the least amount of energy for solvation. In contrast, D-mannose and D-glucose analogues exhibit only slight RI activity with the D-glucose analogue showing slightly increased RI activity (0.00949 mm² at 5.54×10⁻⁶ M). Molar compressibility values correlate nicely with this result. For example D-Galactose analogue 16 has the lowest molar compressibility value and hence, would exhibit the poorest fit into the three-dimensional hydrogen-bonded network of water. Consequently, the energetic cost to incorporate into the three-dimensional hydrogen-bonded network of water would be the largest. This questions the manner in which an AFGP binds to the ice lattice. Biological antifreezes possessing strong TH activity are thought to bind irreversibly to ice via the polypeptide backbone (the driving force being a hydrophobic effect; Sonnichsen, F. D., Sykes, B. D., Davies, P. L., Protein Sci. 1995, 4, 460) and that the carbohydrate moieties are oriented to the water layer of the ice-protein interface. In contrast, early speculation suggested that the hydroxyl groups of the carbohydrate directly bind to the ice lattice.

Due to this apparent correlation between carbohydrate stereochemistry and hydration, but without wishing to be bound by any theory, recrystallization-inhibitors such as biological antifreezes or the C-linked, S-linked, N-linked and O-linked AFGP analogues described herein may function by disturbing the highly ordered structure of supercooled water. This in turn would increase the energy associated with a water molecule transferring from bulk water to the “quasi-liquid layer” (QLL), and subsequently from the QLL to the ice lattice. The QLL is a transitional domain existing at the ice/water interface. While the thickness of the QLL has been shown to be temperature dependant, the characteristics of this region more closely resemble that of the ice lattice (i.e. less dynamic and more ordered than bulk water) at temperatures less than −1° C. The net result of disrupting the ordered structure of supercooled water would be an inhibitory effect on ice growth.

Ice recrystallization is defined as the formation of larger crystals at the expense of smaller ones, and likely occurs when the QLLs of two crystals are in contact with each other (Kingery, J. Appl Phys. 1959, 30, 301). The mechanism of recrystallization has been dealt with extensively in the metallurgical literature. There are two generally accepted theories describing the mechanism of recrystallization: Ostwald ripening and agglomeration (Pronk, P., Infante Ferreira, C. A., Witkamp, G. J. J. Crystal Growth. 2005, 275, e1355; Huige, N J J, Thijsenn, H A C. J. Cryst Growth, 1972, 13/14, 483; Inada, T., Modak, P. R., Chem. Eng. Sci. 2006, 61, 3149). While either mechanism is plausible, the non-uniform crystal shapes from the present RI experiments suggest agglomeration may be the dominant mechanism in this case. According to IR studies by Sadtchenko (Sadtchenko, V., Ewing, G. E., J. Chem. Phys. 2002, 116, 4686), the thickness of the QLL is inversely proportional to temperature, and at −6° C., the thickness of the quasi-liquid layer is ˜1 nm, which equates to about only three monolayers of water. The consequence of such a thin layer means that a carbohydrate residue with low molar compressibility will have a more pronounced affect on the ordering of the bulk water/QLL interface and ultimately ice growth.

C-Linked AFGP analogue 19 possesses custom-tailored antifreeze activity that has potent RI activity and no TH activity. The observed RI activity of C-linked AFGP analogue 19 is a property unique to biological antifreezes (Eniade, A., Purushotham, M., Ben, R. N., Wang, J. B., Horwath, K., Cell Biochem. Biophys. 2003, 38, 115) and PBS negates any false positive effects (Knight, C. A., Hallett, J., DeVries, A. L., Solute Effects on Ice Recrystallization: An Assessment Technique. Cryobiology 1988, 25, 55). Some of the other C-linked analogues have similar activity profiles. While the present results do not elucidate the key structural moieties in the C-linked analogues that are directly responsible for the ice binding event it is likely that the polypeptide backbone is binding to ice.

### C-Linked Carbohydrate:

A general and efficient synthetic strategy for the preparation of structurally diverse C-linked AFGP analogues of homogenous molecular weights has been reported (Anisuzzaman, A. K. M. A., L.; Navia, J. L., Carbohydrate Res. 1998, 174, 265; Filira, F. B., L.; Scolaro, B.; Foffani, M. T.; Mammi, S.; Peggion, E.; Rocchi, R, Int. J. of Biol. Macromol. 1997, 12, 41; Meldal, M. J., K. J., J. Chem. Soc., Chem. Commun. 1990, 483). This methodology is highly attractive in that it is amenable to the high throughput synthesis of complex glycoconjugates. While the methodology has been extended to produce C-linked AFGP analogues of 1.5 to 5.0 kDa, first generation analogues ranged in molecular weight from approximately 1.5 kDa to 4.1 kDa (FIG. 6).

Relative to the native system, several structural modifications were made to produce the instant AFGP analogues. Firstly, the native disaccharide was replaced with the monosaccharide β-D-galactose. Secondly, both alanine residues in the native polypeptide backbone have been replaced with glycine. Thirdly, the L-threonine residue has been replaced with L-lysine. The latter modification is justified based upon the fact that L-threonine is known to be substituted with L-arginine in AFGP 7-8 (Tsuda, T. N., S.-I., Chem. Commun. 1996, 2779) and weak antifreeze protein-specific activity of an L-alanine-L-lysine rich polypeptide was more recently described (Burkhart, F. H., M.; Kessler, H., Angew. Chem. Int. Ed. Engi. 1997, 36, 1191).

Replacement of the labile anomeric carbon-oxygen bond deserves further comment. One of the main reasons to utilize C-linked glycoconjugates is that they possess increased stability. Indeed, there is much precedent in the bioorganic literature where C-linked glycoconjugates have been successfully utilized as probes to investigate various biological processes such as cell surface interactions, adhesion, pathogenesis and fertilization (Debenham, S. D. D., J. S.; Burk, M. T.; Toone, E. J, J. Am. Chem. Soc. 1997, 119, 9897; Ben, R. N. O., A.; Arya, P., J. Org. Chem. 1998, 63, 4817; Ravishankar, R. S., A.; Vijayan, M.; Lim, S.; Kishi, Y., J. Am. Chem. Soc. 1998, 120, 11297; Wang, J. K., P.; Sinay, P.; Gluademans, C. P. J, Carbohydrate Res. 1998, 308, 191). Although these unnatural glycoconjugates are often capable of adapting many more conformations than the native O-linked system, they still bind to the native substrates with similar affinities (Komatsu, S. K. D., A. L.; Feeney, R. E., J. Biol. Chem. 1970, 245, 2909; Feeney, R. E. Y., Y., Adv. Protein Chem. 1978, 32, 191). While it has been suggested that this is not likely a general phenomenon (Hanson, H. C. H., S.; Finne, J.; Magnusson, G., J. Am. Chem. Soc. 1997, 119, 6974), C-linked glycoconjugates remain attractive alternatives to O-linked glycoconjugates.

### Rigid Orientation of Carbohydrate (Short Chain or Amide Bond):

AFGPs are also potent recrystallization inhibitors. While the mechanism by which this re-organization of ice crystals is not known, this property has many potential applications in cryomedicine and the prevention of cellular damage during freezing and thawing cycles. Unfortunately, two factors have precluded the commercialization of native AFGP for medical and industrial applications. These are the limited bioavailability and the inherent instability of the C—O glycosidic bond. Consequently, rationally designed carbon-linked or C-linked AFGP analogues are very attractive. Towards this end, the preparation of C-linked AFGP analogues bearing an amide bond in the side chain have been reported which demonstrate antifreeze protein-specific activity. In the following, the synthesis of a series of “simplified” C-linked AFGP analogues lacking the amide bond is described, and the distance between the carbohydrate moiety and peptide backbone is correlated with antifreeze protein specific activity. Many of the structural features in the first-generation analogues have been incorporated into AFGP analogues. For instance, the native disaccharide has been truncated and replaced by a single galactose residue and the alanine residues replaced with glycines.

Recently, several methodologies have been developed to prepare C-glycosyl amino acids including olefin cross metathesis (OCM) and catalytic asymmetric hydrogenation. The former approach is amenable to preparing analogues such as 18 as it requires the readily available vinyl glycine and C-alkenyl galactose derivatives as starting materials.

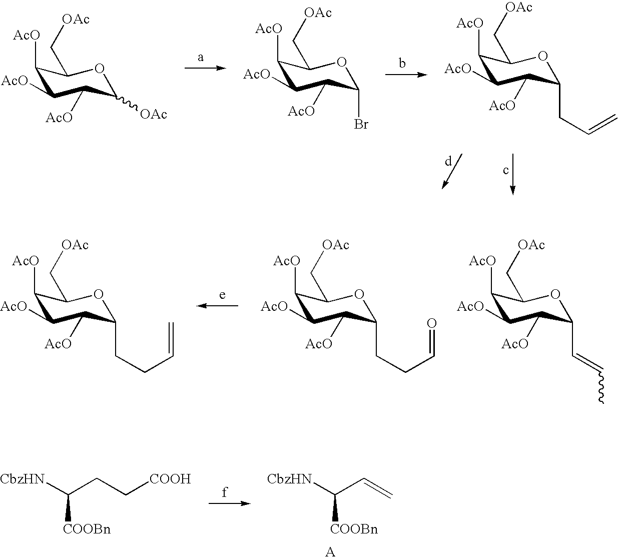

^{a}Reagents and conditions: (a) 30% HBr in AcOH, 4 hrs, 100%; (b) allyl phenyl sulfone, bis(tributyltin), benzene, light, 9 hrs, 90%; (c) PdCl2, benzene, reflux, 60 hrs, 49%; (d) (i) BH3.THF, THF, 0° C., 1.5 hrs; (ii) PCC, CH2Cl2, overnight, 51%; (e) methyl triphenyl phosphonium bromide, tBuOK, ether, 0° C., 1 hr, r.t., overnight, 70%; (f) Pb(OAc)₄, Cu(OAc)2.H2O, benzene, r.t., 1 hr, 90° C., 15 hrs, 34%.

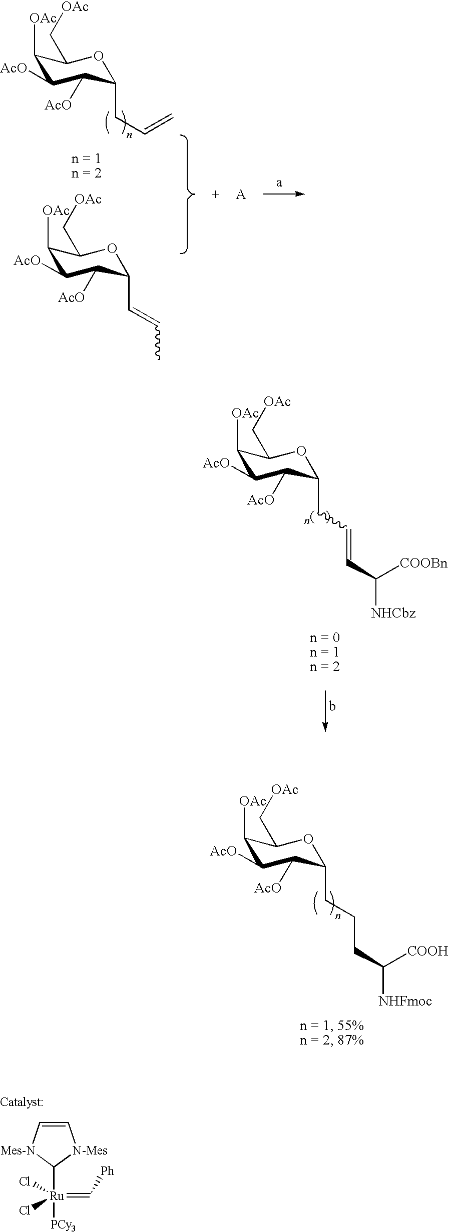

^{a}Reagents and conditions: (a) 20 mol % Grubbs Catalyst (2nd generation), CH₂Cl₂, reflux, 2 days; (b) (i) Pd/C (10%), H2, MeOH, 5 hrs; (ii) Fmoc-OSu, 10% NaHCO3, 1,4-dioxane, 0° C., 1 hr, r.t., overnight.

To prepare the building block for AFGP analogue 18a, an catalytic asymmetric hydrogenation of C-glycosyl enamide ester was adopted. Assembly of building blocks into C-linked AFGP analogues was accomplished by using standard Fmoc-based solid phase synthesis protocols. The protected glycopeptides were cleaved from the resin using TFA and the acetate protecting groups on the pyranose were removed by treatment with sodium in methanol to afford the C-linked AFGP analogues 18a-c (73% to 93% isolated yield) ranging in molecular weight from 1.5 to 1.6 KDa.

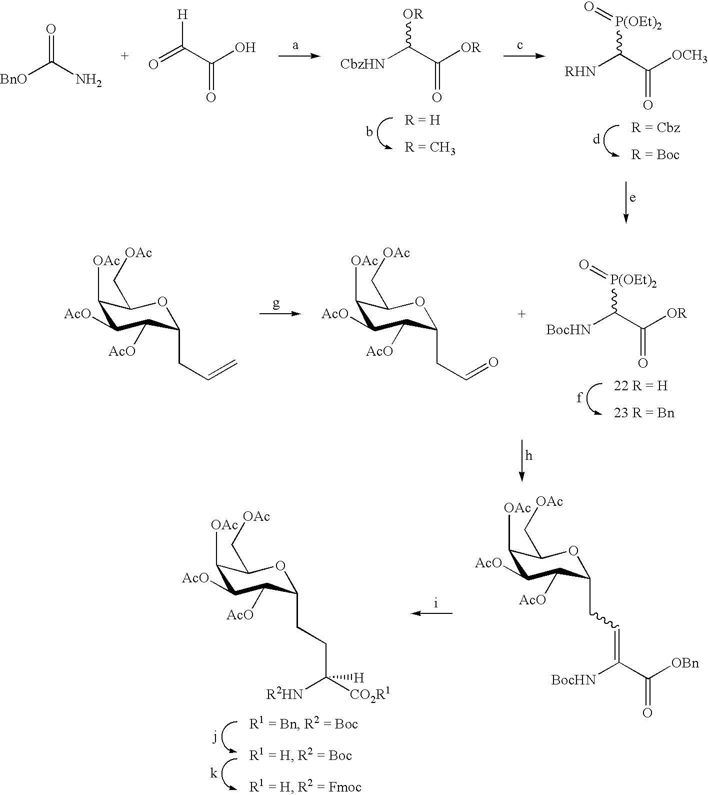

^{a}Reagents and conditions: (a) ether, 16 hrs, 55%; (b) conc. H₂SO₄, MeOH, 2 days, 92%; (c) (i) PCl₃, toluene, 70° C., overnight; (ii) P(OEt)₃, 2 hrs, 66%; (d) (i) H₂, 10% Pd/C. MeOH, 3 hrs; (ii) (Boc)₂O, CH₂Cl₂, overnight, 77%; (e) (i) 2M NaOH, 1,4-dioxane, overnight; (ii) 7% HCl, 86%; (f) BnOH, 4-DMAP, DCC, CH₂Cl₂, overnight, 69%; (g) (i) O₃, CH₂Cl₂, −78° C.; (ii) PPh₃, overnight, 79%; (h) TMG, THF, 1 hr, 80%; (i) [(COD)Rh—((S,S)-Et-DuPHOS)]⁺OTF⁻, 90-100 psi H₂, THF, 2 days, 98%, 69% de; (j) H₂, 10% Pd/C, MeOH, overnight, 89%; (k) (i) 50% TFA/CH₂Cl₂, 0° C., 1 hr; (ii) FmocOSu, 10% NaHCO₃, 1,4-dioxane, 0° C., 1 hr, r.t., overnight, 77%.

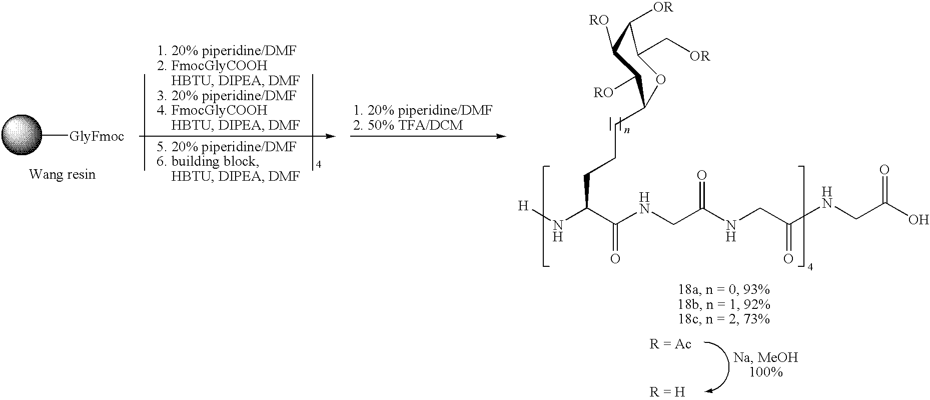

AFGP analogues 18a-c were assayed for antifreeze protein-specific activity using nanoliter osmometry and a recrystallization-inhibition assay. In contrast to the first generation AFGP analogues, 18a-c did not possess any thermal hysteresis or exhibit any dynamic ice shaping abilty.

However, all three analogues demonstrated recrystallization inhibition (RI) activity (FIG. 7) relative to the phosphate-buffered saline (PBS) control. The Y-axis in FIG. 7 represents the mean largest grain size (MLGS) which is an average ice crystal surface area for each sample.

Different concentrations of each analogue in PBS were assayed to rule out the non-specific effects. AFGP analogue 18a was the most potent with activity close to that of native AFGP8. Interestingly, when the side chain length is increased by one or two additional carbon-carbon bonds (18b and 18c), these analogues showed very limited RI activity. The correlation between increased side chain length and decreased RI activity suggests that an optimal distance between the two moieties exists and plays a key role in RI activity of the C-linked analogues. Analogue 18a possesses the same number of atoms between the carbohydrate and peptide moieties as native AFGP. While this analogue is not as potent as AFGP8, it appears to be a more effective recystallization-inhibitor than Type III AFP from the ocean pout (Macrozoarces americanus), which has an effective concentration for RI activity at 7.10×10⁻⁷ M compared with 5.0×10⁻⁸ M in 18a.

### Effectiveness as a Cryoprotectant:

C-Linked AFGP analogue 18a was assessed for its ability to protect WRL-68 cells against cryoinjury during freezing and storage at −25° C. In order to perform this experiment the following protocol was developed.
1) Plate Cells in a clear 96 well, half-area plate. Grow to confluence.
2) Detach Pelletier Unit from Cryobath and turn it on. Change temperature setting to −25° C.
3) Get some crushed ice in at least two Styrofoam containers and put them in the hood with the UV light on for half an hour.
4) Transfer some UW solution to a 10 ml sterile vial (under sterile conditions in the hood). Put the UW bottle back in the fridge.
5) Make enough of a 5 mg/ml solution (in UW from your vial) of your compound to be able to plate two rows at 5 mg/ml, and then make subsequent serial dilutions for all further rows.

### Plate Set-Up:

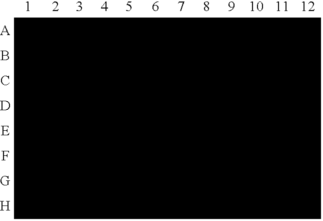

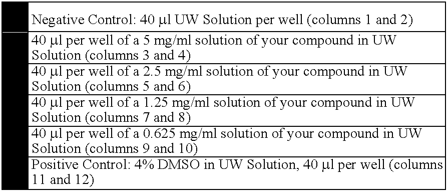

Make the 5 mg/ml solution in a sterile 1.5 ml-2 ml eppendorf tube.
6) Make enough 4% DMSO solution (in another eppendorf tube) to plate the two positive control rows, taking into account loss of solution to the boat.
7) Put your vial with the remaining UW, the tube with the DMSO, and the tube with your compound on crushed ice. Put two empty boats on ice. Also put your cells on crushed ice. Let them all cool for half an hour.
8) Add the required UW solution to the boat. Add the required DMSO solution to the other boat (label them).
9) Spray a few Kim Wipes with ethanol, fold them into a large square and put them in the hood. Shake out the medium that is in the plate onto the Kim Wipes. Replace the empty plate (with cells in it) on the ice to keep the cells cold.
10) Add 40 μl of UW solution to the first two rows with the multi-channel pipette.
11) Add 40 μl of the 5 mg/ml solution to the next two rows, to one well at a time. Use the P100 pipette and add directly from the eppendorf tube to avoid losing any solution.
12) Dilute remaining solution to 2.5 mg/ml, and add to the next two rows in the same fashion. Continue for remaining concentrations.
13) Add 40 μl DMSO to last two rows.
14) CAREFULLY tape your plate shut. Then seal it in three Ziploc bags, removing all of the air. KEEP THE PLATE LEVEL AT ALL TIMES. Tape down loose edges if you want. Be sure bags are sealed well. Try to keep the cells cold and touch the bottom of the plate as little as possible with your hands so the cells do not warm up.
15) Bury the wrapped plate in ice, keeping the plate level.
16) Place the wrapped plate in the cryobath, weighted down with the red weights, for 18 hours. Use the cotton gloves in cell culture with large sized gloves over top.
17) Remove the plate from the bath and unwrap. Allow it to thaw in the hood just until the ice in the wells barely disappears (so that the solution is still cold).
18) Centrifuge at 1200 rpm for 5 minutes to move any detached cells to the bottom.
19) Dump out the UW solution onto a Kim Wipe as described previously, and add 40 μl of pre-warmed MEM (37° C.) to each well (use a boat and the multi-channel pipette).
20) Add 4 μl of MTT solution to each well (use a boat and the smaller volume multi-channel pipette).
21) Shake in the plate reader for 10 seconds at low intensity.
22) Incubate for 2-4 hours.
23) Add 40 μl isopropanol solution to each well. Then go back and aspirate each row ˜200 times to dissolve the purple precipitate. Avoid foaming, and be sure that are no purple crystals left in your tips when you discard your tips. If your have problems with foaming leave the plate (with its lid on) and come back to it later. Once you have added the isopropanol to all wells the cells are dead so the timing on the dissolving is not as important, so long as evaporation is minimized.
24) Read the absorbance at 570 nm.

Note: all materials were kept on ice, vials were not held by hand, nothing is added to the cells that is not at 0° C., and the cells were kept at 0° C.

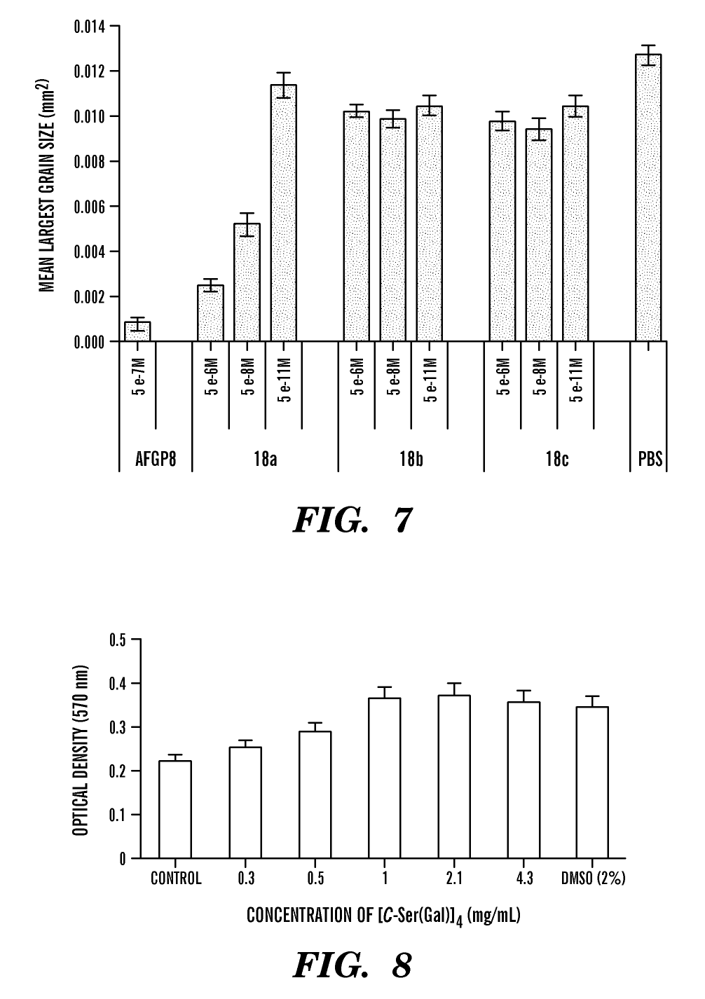

The results of this assay are shown in FIG. 8. This data shows that C-linked Serine analogue 18a exhibits significant protection against croinjury as evidenced by the nearly two-fold increase in cell viability as determined by MTT assay.

In summary, olefin cross metathesis and catalytic asymmetric hydrogenation has been utilized to prepare a series of novel C-linked AFGP analogues with different distances between the carbohydrate and peptide backbone moieties. The analogue with the shortest distance between these moieties is a potent recrystallization inhibitor. Such recrystallization inhibitors have also been shown to possess cryoprotectant activity, and accordingly have wide-spread medical, industrial and commercial applications.

### Experiment 2

### Hydration Index of a Carbohydrate and Correlation to RI Activity

Carbohydrates are prolific in biological systems and are involved in many processes ranging from cellular adhesion, cell signaling, infection and regulation of the immune response (Cheng, C. C.; Bennett, D. Cell 1980, 19, 537-543; Grabel, L. B.; Rosen, S. D.; Martin, G. R. Cell 1979, 17, 477-484; Stanley, P.; Sudo, T. Cell, 1981, 23, 763-769; Neufeld, E.; Ashwell, G. In Biochemistry of Glycoproteins and Proteoglycans. W. J. Lennarz, ed. (New York: Plenum Press), 1980; p 241-266). In addition, carbohydrates are involved in the cryoprotection of organisms inhabiting cold climates (Yeh, Y.; Feeney, R. E. Chem. Rev. 1996, 96, 601).

It is well accepted that hydration of proteins plays an important role in modulating protein function (Frauenfelder, H.; Fenimore, P. W.; McMahon, B. H. Biophys. Chem. 2002, 98, 35-48). Similarly, hydration or solvation of carbohydrates or oligosaccharides temper their biological activity. Despite the undisputed importance of hydration, assessing the degree of hydration is not a trivial process (Quiocho, F. A. Ann. Rev. Biochem. 1986, 55, 287; Lemieux, R. U. Chem. Soc. Rev. 1989, 18, 347). While NMR techniques have reportedly been used to assess hydration of complex oligosaccharides, the results of these studies are often subject to a large degree of uncertainty (Corzana, F.; Motawia, M. S.; Du Penhoat, C. H.; Perez, S.; Tschampel, S. M.; Woods, R. J.; Engelsen, S. B. J. Comput. Chem. 2004, 25, 573; Furó, I.; Pócsik, I.; Tompa, K.; Teeäär, R.; Lippmaa, E. Carbohydr. Res. 1987, 166, 27). In contrast, the hydration of simple carbohydrates has been extensively studied during the last few decades (Franks, F. Pure Appl. Chem. 1987, 59, 1189). These studies have been limited to small temperature and pressure ranges because these molecules possess many different conformations in aqueous solution (Franks, F. Pure Appl. Chem. 1987, 59, 1189; Franks, F.; Lillford, P. J.; Robinson, G. J. Chem. Soc., Faraday Trans. 1 1989, 85, 2417); nonetheless, they demonstrate that carbohydrate hydration is closely correlated to stereochemistry. While the exact reasons for this are the subject of much debate, various hypotheses have been proposed that rationalize hydration characteristics and the subsequent influence of hydration on bulk water (Galema, S. A.; Høiland, H. J. Phys. Chem. 1991, 95, 5321-5326; Galema, S. A.; Eduardo, H.; Engberts, J. B. F. N.; Raul Grigera, J. Carbohydr. Res. 1994, 265, 215; Galema, S. A.; Engberts, J. B. F. N.; Høiland, H.; Førland, G. M. J. Phys. Chem. 1993, 97, 6885; Stokes, R. H.; Robinson, R. A. J. Phys. Chem. 1966, 70, 16; Suggett, A.; Ablett, S.; Lillford, P. J.; J. Solution Chem. 1976, 5, 17; Tait, M. J.; Suggett, A.; Franks, F.; Abblett, S.; Quikenden, P. A. J. Solution Chem. 1972, 1, 131; Uedaira, H. J. Solution Chem. 1985, 14, 7; Kabayama, M. A., Patterson, D., Piche, L., Can. J. Chem. 1958, 36, 557; Miyajima, K., Machida, K., Nagagaki, M., Bull. Chem. Soc. Jpn. 1985, 58, 2595; Walkinsaw, M. D., J. Chem. Soc., Perkin Trans. 2 1987, 1903; Danford, M. D. J. Am. Chem. Soc. 1962, 84, 3965; Warner, D. T. Nature 1962, 196, 1055; Franks, F., Cryobiology 1983, 20, 335; Suggett, A., J. Solution Chem. 1976, 5, 33).

The present inventors have successfully designed functional C-linked antifreeze glycoprotein (AFGP) analogues possessing custom-tailored antifreeze activity. These compounds are useful as inhibitors of recrystallization and do not possess thermal hysteresis (TH) activity. This is significant as studies demonstrate that cellular damage as a result of recrystallization is the major cause of decreased cellular viability upon cryopreservation (Wang, T.; Zhu, Q.; Yang, X.; Layne, J. R.; DeVries, A. L. Biopolymers 1994, 31, 185; Petzel, D. H.; DeVries, A. L. Cryobiology 1977, 16, 585). As such, these compounds are useful as cryoprotectants for cryomedical and commercial applications, especially in applications where improved cryoadjuvants and cryopreservation protocols are required such as for preserving donor organs (Hafez, T.; Fuller, B. Advances in Biopreservation. 2007, 197).

The present inventors have shown that hydration of a C-linked carbohydrate moiety in a C-linked AFGP analogue is a contributing factor to antifreeze activity, specifically recrystallization-inhibition (RI) activity. The experiments of Example 2 explore in depth the relationship between hydration of carbohydrates and carbohydrate derivatives with recrystallization-inhibition activity.

### Material and Methods

The monosaccharides used in this study were commercially available and purchased from Sigma-Aldrich. All C-linked pyranose derivatives were synthesized using standard literature procedures (Czechura, P., Tam, R. Y., Murphy, A. V., Dimitrijevic, E., and Ben, R. N. J. Am. Chem. Soc. 2008, 130, 2928-2929). Isentopic molar compressibility (IMC) values were obtained from ultrasound density measurement as reported in the literature (Galema, S. A., and Høiland, H. J. Phys. Chem. 1991, 95, 5321-5326). Hydration numbers, nₕ, were obtained using the Passynsky equation, Eq.1 (Shiio, H. J. Am. Chem. Soc. 1958, 80, 70; Moulik, S. P.; Gupta, S. Can. J. Chem. 1989, 67, 356; Bockris, J. O. M.; Reddy, A. K. N. Modern Electrochemistry; Plenum: New York, 1977; Vol. I, p 127; Ernst, S.; Jezowska-Trzebiatowska, B. J. Phys. Chem. 1975, 79, 2113):

nₕ=(n_{w}/nₛ)(1−βₛ/βₛₒ) (Eq.1)

where n_{w}, and nₛ are the mole fractions of water and the solute, respectively; βₛ and βₛₒ are the isentropic coefficients of compressibility of the solute and water, respectively.

### Recrystallization-Inhibition (RI) Assay:

Sample analysis for RI activity was performed using the “splat cooling” method as described previously (Knight, C. A.; Hallet, J.; DeVries, A. L. Cryobiology, 1998, 25, 55). In this method, the analyte is dissolved in phosphate buffered saline (PBS) solution and a 10 μL drop of this solution is dropped from a micropipette through a two metre high plastic tube (10 cm in diameter) onto a block of polished aluminum pre-cooled to approximately −80° C. The droplet freezes instantly on the polished aluminum block and is approximately 1 cm in diameter and 20 μm thick. This wafer is then carefully removed from the surface of the block and transferred to a cryostage held at −6.4° C. for annealing. After a period of 30 minutes, the wafer was photographed between crossed polarizing filters using a digital camera (Nikon CoolPix 5000) fitted to the microscope. A total of three images are taken from each wafer. During flash freezing, ice crystals spontaneously nucleate from the super-cooled solution. These initial crystals are relatively homogenous in size and quite small. During the annealing cycle recrystallization occurs, resulting in a dramatic increase in ice crystal size. A quantitative measure of the difference in recrystallization inhibition of two compounds X and Y is the difference in the dynamics of the ice crystal size distribution. In other words, X shows greater RI activity than Y if the crystals in X grow more slowly than in Y. If on average, crystals in X are growing more slowly than in Y, at any given fixed time the average crystal size in X will be smaller than in Y and one can obtain a quantitative measure of RI by characterizing the distribution of crystal sizes. Image analysis of the ice wafers was performed using novel domain recognition software (DRS) (Jackman, J.; Noestheden, M.; Moffat, D.; Pezacki, J. P.; Findlay, S.; Ben, R. N. Biochemical and Biophysical Research Communications 2007, 354, 340) program that was developed at the Steacie Institute for Molecular Sciences (SIMS) of the National Research Council of Canada (NRCC). This processing employed of the Microsoft Windows Graphical User Interface to allow a user to visually demarcate and store the vertices of ice domains in a digital micrograph. These data were then used to calculate the domain areas. To eliminate the need to fully process each micrograph, an algorithm was developed to randomly display a number of x/y locations. The algorithm made use of a built-in pseudo random number generator (rand(x)) and was written so that no two locations were closer than 1/10th the field of view of the micrograph. The formula for the area of a polygon that is not self-intersecting and contains no holes is then given by Eq.2,

$$A = \frac{1}{2} {\sum }_{i = 0}^{N - 1} ( {x}_{i } {y}_{i + 1} - {x}_{i + 1} {y}_{i} ) \qquad ( Eq . 2. )$$

where N is the number of vertices, and x₀, y₀ to x_{N-1}, y_{N-1} are the vertices circumventing the polygon in a clockwise direction. The point x₀, y₀ is assumed to be equivalent to the point x_{N}, y_{N}. The software was written in C using Microsoft Visual Studio 6.0 on a Pentium class personal computer running Microsoft Windows 2000 or XP. All data was plotted and analyzed using Microsoft Excel.

### Thermal Hysteresis Assay:

Nanoliter osmometry was performed using a nanoliter osmometer (Clifton Technical Physics, Hartford, N.Y.) as described by Chakrabartty and Hew (Chakrabartty, A.; Hew, C. L. Eur. J. Biochem. 1991, 202, 1057-1063). All measurements were made in doubly distilled water. Ice crystal morphology was observed through a Leitz compound microscope equipped with an Olympus 20× (infinity corrected) objective, Leitz Periplan 32× photo eyepiece and a Hitachi KP-M2U CCD camera connected to a Toshiba MV13K1 TV/VCR system. Still images were captured directly using a Nikon CoolPix digital camera.

### Results and Discussion

The “hydration layer” is defined as water that encompasses the carbohydrate and is often bound very tightly. Specific hypotheses to rationalize the observed hydration characteristics of a carbohydrate include: hydration number (Stokes, R. H.; Robinson, R. A. J. Phys. Chem. 1966, 70, 16; Suggett, A.; Ablett, S.; Lillford, P. J.; J. Solution Chem. 1976, 5, 17; Tait, M. J.; Suggett, A.; Franks, F.; Abblett, S.; Quikenden, P. A. J. Solution Chem. 1972, 1, 1311 Uedaira, H. J. Solution Chem. 1985, 14, 7), anomeric effect (Kabayama, M. A., Patterson, D., Piche, L., Can. J. Chem. 1958, 36, 557), hydrophobic index (Miyajima, K., Machida, K., Nagagaki, M., Bull. Chem. Soc. Jpn. 1985, 58, 2595), hydrophilic volume (Walkinsaw, M. D., J. Chem. Soc., Perkin Trans. 2 1987, 1903), and compatibility with bulk water based upon the position of the next-nearest-neighbour hydroxyl groups (Danford, M. D. J. Am. Chem. Soc. 1962, 84, 3965; Warner, D. T. Nature 1962, 196, 1055). Subsequent to the latter hypothesis, a revised stereospecific hydration model has suggested that hydration of a carbohydrate depends upon the ratio of axial to equatorial hydroxyl groups (Franks, F., Cryobiology 1983, 20, 335; Suggett, A., J. Solution Chem. 1976, 5, 33). In an attempt to generate a unifying hypothesis consistent with all of the above, key thermodynamic parameters thought to dictate hydration were measured by Galema et al. in an attempt to rationalize the influence of carbohydrate stereochemistry (Galema, S. A.; Hølland, H. J. Phys. Chem. 1991, 95, 5321-5326; Galema, S. A.; Eduardo, H.; Engberts, J. B. F. N.; Raul Grigera, J. Carbohydr. Res. 1994, 265, 215; Galema, S. A.; Engberts, J. B. F. N.; Høiland, H.; Førland, G. M. J. Phys. Chem. 1993, 97, 6885). They studied the hydration of monosaccharides using molecular dynamics simulations, kinetic experiments, and density and ultrasound measurements. Subsequently, the partial molar volumes, isentropic partial molar compressibilities, and hydration numbers of many commercially available hexoses have been determined and correlated to carbohydrate stereochemistry. Table 1 reports the isentropic molar compressibility values and hydration numbers for various mono- and disaccharides (Galema, S. A.; Høiland, H. J. Phys. Chem. 1991, 95, 5321-5326; Galema, S. A.; Eduardo, H.; Engberts, J. B. F. N.; Raul Grigera, J. Carbohydr. Res. 1994, 265, 215; Galema, S. A.; Engberts, J. B. F. N.; Høiland, H.; Førland, G. M. J. Phys. Chem. 1993, 97, 6885).

TABLE 1

| | Molar | |
|---|---|---|
| | Compress- | |
| | ibility | |
| | (K₂°(s)x10⁴, | |
| | cm³ mol⁻¹ | Hydration |
| Carbohydrate | bar⁻¹) | Number |
| 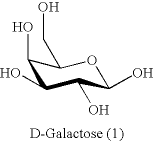 | −20.8 −20.4 | 8.7 |
| 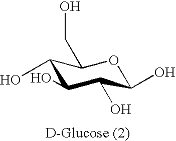 | −17.6 | 8.4 |
| 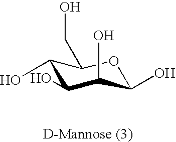 | −16.0 | 8.1 |
| 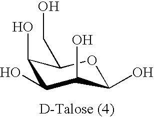 | −11.9 | 7.7 |
| 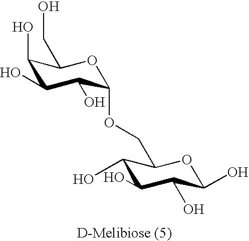 | −31.2 | 15.5 |
| 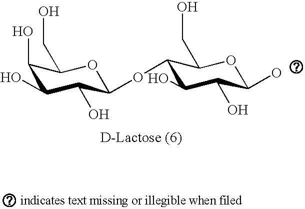 | −31.1 −30.4 | 15.3 |
| 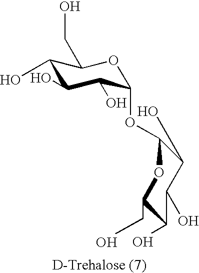 | −30.2 | 15.3 |
| 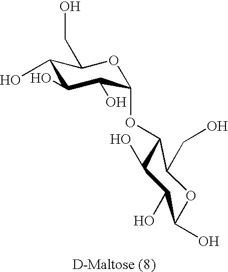 | −23.7 | 14.5 |
| 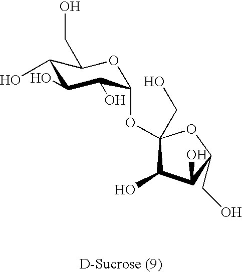 | −17.8 | 13.9 |

Isentropic molar compressibility (K₂°(s)x10⁴, cm³ mol⁻¹ bar⁻¹) and Hydration Numbers of Various Monosaccharides, 1-4, and Disaccharides, 5-9 (Galema, S.A.; Høiland, H. J. Phys. Chem. 1991, 95, 5321-5326; Høiland, H.; Holvik, H. J. Solution Chem. 1978, 7, 587; Shahidi, F.; Farrell, P. G.; Edward, J. T. J. Solution Chem. 1976, 5, 807).

[symbol]

[symbol]

indicates data missing or illegible when filed

As an extension and application of this stereospecific hydration model, Furuki has measured the heat of fusion of ice in carbohydrate solutions and subsequently determined the amount of unfrozen water (U_{w}) surrounding the carbohydrate molecule using differential scanning calorimetry (DSC) (Furuki, T. Carbohydr. Res. 2000, 323, 185-191; Furuki, T. Carbohydr. Res. 2002, 337, 441-450). These studies suggested that larger amounts of unfrozen water are correlated with poorer compatibility of the sugar with the three dimensional hydrogen-bonded network of bulk water; thus, it was proposed that the antifreeze activity of carbohydrates is a function of hydration, which in turn is dependant upon carbohydrate stereochemistry. The primary criterion for antifreeze activity in this study was non-colligative freezing point depression as determined by DSC. While DSC has been utilized to study thermal hysteresis (TH) activity in AFPs and AFGPs, the more conventional technique is nanolitre osmometry (Yeh, Y.; Feeney, R. E. Chem. Rev. 1996, 96, 601. However, neither of these techniques assesses recrystallization-inhibition (RI). TH is defined as a selective depression of the freezing point of a solution relative to a static melting point, whereby the difference between these two temperatures is known as a thermal hysteretic gap. The significance of this gap is that within this temperature range, a seeded ice crystal is in a stable equilibrium and does not overgrow, resulting in a non-frozen solution. Thermal hysteresis is always preceded by dynamic ice shaping (DIS), which is a direct result of a solute binding at the interface of the ice lattice and the quasi-liquid layer (QLL). Alternatively, recrystallization inhibition activity prevents (or slows down) the enthalpically driven re-organization of individual ice crystals in an already frozen sample (Yeh, Y.; Feeney, R. E. Chem. Rev. 1996, 96, 601; Knight, C. A.; Duman, J. G. Cryobiology 1986, 23, 256-262; Knight, C. A.; Hallett, J.; DeVries, A. L. Cryobioogy, 1988, 25,55; McKown, R. L.; Warren, G. J. Cryobiology 1991, 28, 474; Yeh, Y.; Feeney, R. E.; McKown, R. L.; Warren, G. J. Biopolymers 1994, 34, 1495). While the mechanism of TH activity remains the source of much debate Hew C. L.; Yang, D. S. C. Eur. J. Biochem. 1992, 203, 33), it is recrystallization inhibition that is the most desirable property of a cryoprotectant as the majority of cellular damage from cryopreservation occurs during the holding and thawing phase of preservation where recrystallization is a dominant process (Knight, C. A.; Duman, J. G. Cryobiology 1986, 23, 256-262.; Mazur, P. C. Science 1970, 168, 939; Mazur, P. C. Am. J. Physiol. 1984, 247, C125-C142).

### Assessing the Carbohydrate-Ice Interaction:

Recent reports have suggested that the antifreeze activity of simple mono- and disaccharides is based on complimentarily of hydroxyl groups with the ice lattice. It has been proposed that the optimal distance between hydroxyl groups is 4.2-4.5 Angstroms (Baruch, E.; Belostotskii, A. M.; Mastai, Y. J. Mol. Struct. 2008, 874, 170). To investigate this possibility we examined two monosaccharides (galactose and glucose) and two disaccharides (lactose and trehalose) for antifreeze activity as a function of thermal hysteresis using nanoliter osmometry (Chakrabartty, A.; Hew, C. L. Eur. J. Biochem. 1991, 202, 1057-1063. From Table 2 it is evident that none of these carbohydrates exhibit dynamic ice shaping, and therefore do not have thermal hysteresis activity. The slight freezing point depressions are due to the colligative properties of each carbohydrate. More interesting is the fact that none of these compounds display any degree of DIS and that the ice crystal morphologies are consistent with those of samples of ice in water.

TABLE 2
Ice crystal morphology and melting points of 10 mg/mL solutions of
D-galactose, D-glucose, D-melibiose and D-trehalose in double-distilled
water, using nanolitre osmometry.

| | Melting | Crystal |
|---|---|---|
| Carbohydrate | Point (° C.) | Morphology |
| 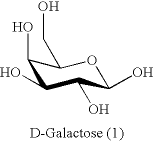 | −0.069 | 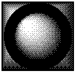 |
| 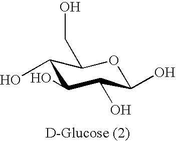 | −0.055 | 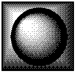 |
| 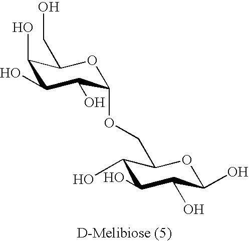 | −0.17 | 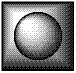 |
| 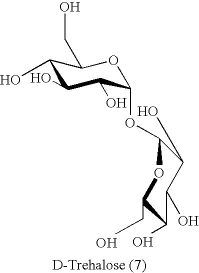 | −0.015 | 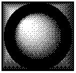 |

Without wishing to be bound by any theory, this finding is significant as it indicates that no direct interaction with the ice lattice is occurring as previously proposed (Baruch, E.; Belostotskii, A. M.; Mastai, Y. J. Mol. Struct. 2008, 874, 170), and that the proposed optimal distance between hydroxyls of 4.2-4.5 Angstroms is not the only factor responsible for imparting antifreeze activity. This questions the hypothesis that complimentarity of hydroxyl groups with the ice lattice is necessary for antifreeze activity. While it has been suggested that the protein-ice interaction has an element of surface complimentarity (Leinala, E. K.; Davies, P. L.; Jia, Z. Structure 2002, 10, 610), it is still unclear how biological antifreezes recognize the pseudo-ordered quasi-liquid layer (QLL) separating the ice lattice from bulk water. Recent molecular dynamic simulations have implied that the QLL plays an important role in the binding of biological antifreezes to ice (Madura, J. D.; Baran, K.; Wierzbicki, A. J. Mol. Recognit. 2000, 13, 101). Herein it is proposed that a key contributing factor for antifreeze activity is hydration of the carbohydrate residue. Given that binding of a solute to ice is not a prerequisite for RI activity, a variety of carbohydrates were examined for RI as a function of hydration numbers.

### The Effect of Concentration on Recrystallization Inhibition Activity:

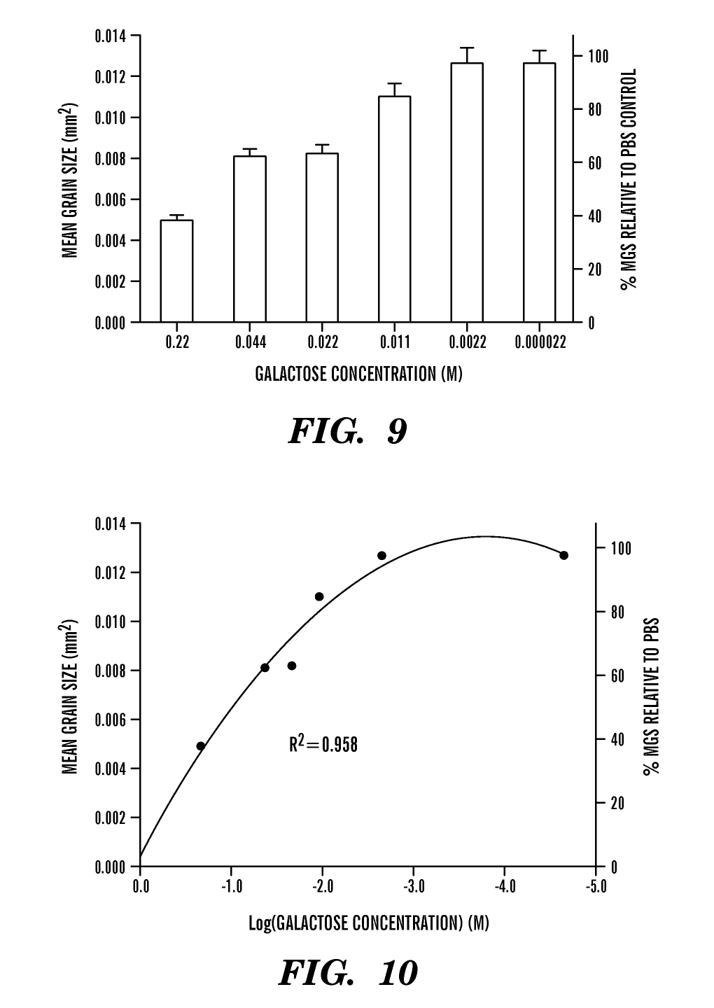

To determine the optimal working concentration for our RI assay, a concentration scan was performed. FIG. 9 shows that a 0.22 M solution of galactose in PBS is an effective inhibitor of recrystallization. While this concentration results in reasonable RI activity, the viscosity of this solution was quite high and posed technical difficulties that adversely affected assay performance. A 0.044 M solution showed RI activity similar to that of a 0.022 M solution. Given the difficulties associated with the viscosity of 0.22 M galactose solution and the fact that there was no statistically significant difference (Student's T-test was performed at 95% confidence level) between 0.044 M and 0.022 M solutions of galactose, concentrations of 0.022 M were employed for all saccharides. The overall relationship between carbohydrate concentration and RI activity appears to be a logarithmic relationship as shown in FIG. 10. This suggests that the RI activity of galactose is non-colligative in nature, which is similar to the thermal hysteresis activity observed in AFGP-8 (Bouvet, V.; Lorello, G.; Ben, R. N. Biomacromolecules. 2006, 7, 565). This non-colligative relationship has also been reported by Uchida, who utilized field-emission type transmission electron microscopy (FE-TEM) to study ice crystal size as a function of trehalose concentration (Uchida, T.; Nagayama, M.; Shibayama, T.; Gohara, K. J. Crys. Growth 2007, 299, 125).

### Carbohydrate Stereochemistry:

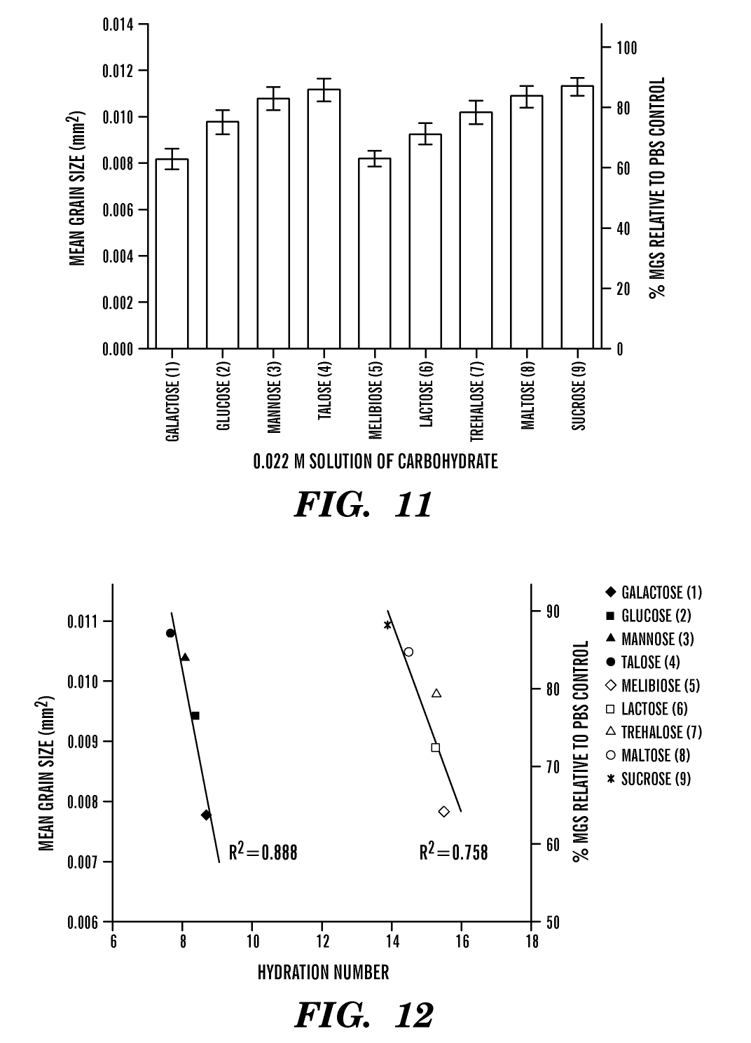

The RI activity of simple mono- and disaccharides was assessed to better understand the relationship between hydration as a function of hydroxyl stereochemistry and RI activity (FIG. 11). Previous reports suggested that the compatibility of a carbohydrate with the three-dimensional hydrogen-bonded network of water is governed by carbohydrate stereochemistry (Galema, S. A.; Høiland, H. J. Phys. Chem. 1991, 95, 5321-5326; Galema, S. A.; Eduardo, H.; Engberts, J. B. F. N.; Raul Grigera, J. Carbohydr. Res. 1994, 265, 215; Galema, S. A.; Engberts, J. B. F. N.; Høiland, H.; Førland, G. M. J. Phys. Chem. 1993, 97, 6885; Dashnau, J; Sharp, K. A.; Vanderkooi, J. M. J. Phys. Chem. B 2005, 109, 24152) and this is consistent with experimentally derived molar compressibility values which vary as a function of hydroxyl stereochemistry at C2 and C4 (Table 1).

As is evident from FIG. 11, the trend in RI activity for each class of carbohydrates, 1-4 (monosaccharides) and 5-9 (disaccharides) correlates positively with isentropic molar compressibility values and hydration numbers (Table 1), suggesting that hydration as a function of carbohydrate stereochemistry has a significant influence on RI activity. This is consistent with our data on the RI activity of various C-Linked AFGP analogues whereby analogues containing carbohydrates with low isentropic molar compressibilities (such as galactose) are poorly hydrated and hence are effective inhibitors of recrystallization. While this hypothesis was based upon isentropic molar compressibilities, which is the change in volume of disturbed water by the addition of a solute molecule, this measurement of solute hydration may not be truly representative of the actual hydration state.

Many experimental methods have been reported to study the structure of solutions. These range from near infrared spectrophotometry (Hollenberg, J. L.; Hall, D. 0. J. Phys. Chem. 1983, 87, 695), density and density ultrasound (Galema, S. A.; Høiland, H. J. Phys. Chem. 1991, 95, 5321-5326; Branca, C.; Magazii, S.; Maisano, G.; Migliardo, F.; Migliardo, P.; Romeo, G. J. Phys. Chem. B, 2001, 105, 10140), nuclear magnetic and dielectric-relaxation (Uedaira, H.; Uedaira, H. Cellular and Molecular Biology, 2001, 47, 823-829; Matsuoka, T.; Kada, T.; Murai, K.; Koda, S.; Nomura, H. J. Mol. Liq. 2002, 98-99, 319), quasi elastic neutron scattering (Magazù, S.; Villari, V.; Migliardo, P.; Maisano, G.; Telling, M. T. F. J. Phys. Chem. B 2001, 105, 1851), terahertz spectroscopic measurement (Heyden, M.; Briindermann, E.; Heugen, U.; Niehues, G.; Leitner, D. M.; Havenith, M. J. Am. Chem. Soc. 2008, 130, 5773), and viscosity and acoustic measurements (Branca, C.; Magazù, S.; Maisano, G.; Migliardo, F.; Migliardo, P.; Romeo, G. J. Phys. Chem. B, 2001, 105, 10140; Mathlouthi, M.; Hutteau, F. Food Chemistry, 1999, 64, 77-82), to molecular dynamic simulations (Engelsen, S. B.; Monteiro, C.; de Penhoat, C. H.; Perez, S. Biophysical Chemistry 2001, 93, 103). The density ultrasound technique is regarded as one of the best experimental methods in that it delineates subtle differences in solution structure, such as water that is tightly bound to a solute versus water that is only loosely associated with a solute (Gharsallaous, A.; Roge, B.; Genotelle, J.; Mathlouthi, M. Food Chem. 2008, 1061443-1453). While this may be true to obtain accurate adiabatic compressibility coefficients, recent studies have shown that hydration numbers calculated from these coefficients represent a more accurate summary of contributions influencing hydration of the solute molecule (Gliński, J.; Burakowski, A. Eur. Phys. J. Special Topics 2008, 154, 275). The hydration number differs from isentropic molar compressibility values in that hydration numbers accurately predict the total number of water molecules hydrogen-bonded to the sugars. However, this is still regarded as a dynamic measurement because it really predicts the number of water molecules that have a relatively long residence time and hence move in solution with the sugars. Given that hydration numbers are a more accurate representation of solute hydration, we obtained literature hydration values for the mono- and disaccharides in Table 1 and plotted these as a function of R¹ activity (FIG. 12). It is important to note that the hydration number of each carbohydrate is calculated using the respective isentropic molar compressibility coefficient (Galema, S. A.; Høiland, H. J. Phys. Chem. 1991, 95, 5321-5326).

From this data, two observations are evident. Firstly, each series of carbohydrates fits a linear relationship with R² values of 0.889 and 0.758 for mono- and disaccharides, respectively. Secondly, a predicted increase in RI activity for the disaccharides relative to monosaccharides was not observed. For example, the large difference in hydration numbers between monosaccharide galactose (8.7) and disaccharide melibiose (15.5) does not result in a corresponding increase in RI activity. More surprisingly, the difference in hydration numbers between galactose and sucrose (13.9) resulted in a decrease in R¹ activity for the latter. We believe this is due to the difference in steric volume between monosaccharides and disaccharides. Hydration numbers were derived according to the Passynsky equation (Eq. 1), using molar compressibility coefficients, β, which were obtained from ultrasound and density measurements (Eq. 3) (Galema, S. A.; Høiland, H. J. Phys. Chem. 1991, 95, 5321-5326),

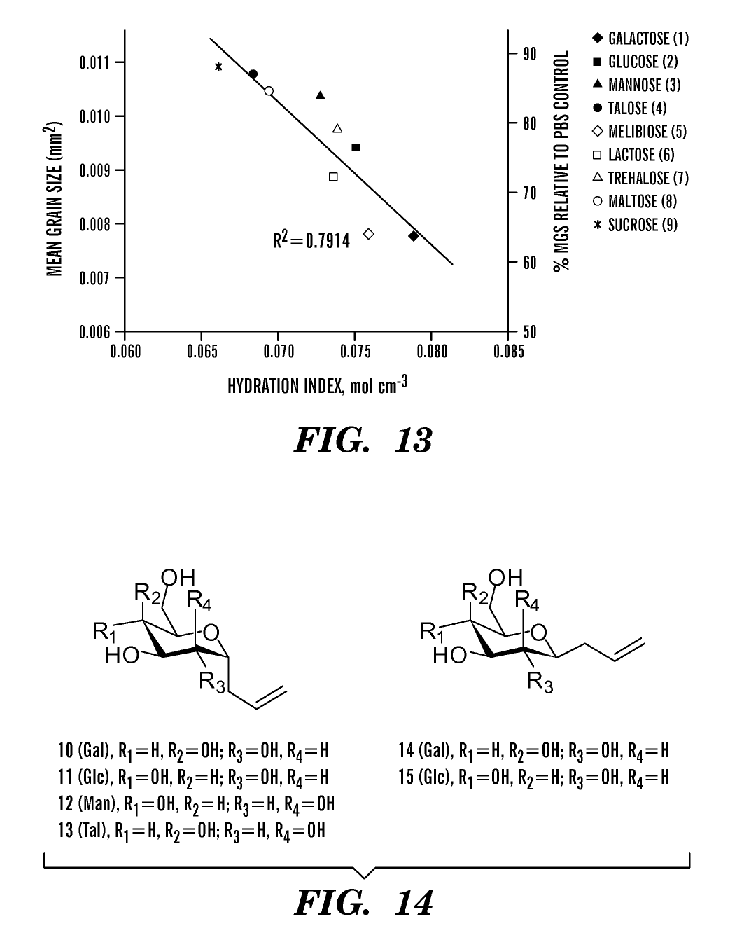

β=1/u²d (Eq. 3)

where u is the speed of sound, and d is the density of the solution. Dividing hydration numbers by partial molar volumes (Høiland, H.; Holvik, H. J. Solution Chem. 1978, 7, 587; Shahidi, F.; Farrell, P. G.; Edward, J. T. J. Solution Chem. 1976, 5, 807) results in a description of the number of tightly bound water molecules per molar volume of carbohydrate. We refer to this value as a hydration index. Similar correlations have been reported by Parke in relating the taste properties of solutes of various masses, volumes, and hydrophobicity to the hydration per volume of the solute (Parke, S. A.; Birch, G. G.; Dijk, R. Chem. Senses 1999, 24, 271). FIG. 13 shows that plotting the hydration index against R¹ activity of carbohydrates 1-9 results in a single communal correlation for all carbohydrates with an R² value of 0.795, compared to FIG. 12 which had two distinct correlations. This suggests that the absolute number of hydrated water molecules which surround a carbohydrate is not the only influence for RI activity, but rather it is how concentrated the water molecules are per unit of solute.

### RI Activity Comparison Between C-Glycosides and O-Glycosides

Our synthesized AFGP analogues are C-linked in nature. While the conformation of C-linked carbohydrate analogues is generally accepted to be similar to O-linked systems (Espinosa, J. F.; Montero, E.; Vian, A.; Garci´a, J. L.; Dietrich, H.; Schmidt, R. R.; Martín-Lomas, M.; Imberty, A.; Cañada, F. J.; Jiménez-Barbero, J. J. Am. Chem. Soc. 1998, 120, 1309; Wang, J.; Kova´ĉ, P.; Sinay- [symbol] , P.; Glaudemans, C. P. J.; Carbohydr. Res. 1998, 308, 191; Wang, Y.; Barbirad, S. A.; Kishi, Y.; J. Org. Chem. 1992, 57, 4681 Ravishankar, R.; Surolia, A.; Vijayan, M.; Lim, S.; Kishi, Y. J. Am. Chem. Soc. 1998, 120, 11297; Ma, B.; Schaefer, H. F., III; Allinger, N. L. J. Am. Chem. Soc. 1998, 120, 3411), the relationship between molar compressibility and/or hydration and RI activity has not been previously studied. It has been suggested that hydration is dictated predominately by the substituents at C2 and C4 of the pyranose ring (Galema, S. A.; Høiland, H. J. Phys. Chem. 1991, 95, 5321-5326; Ma, B.; Schaefer, H. F., III; Allinger, N. L. J. Am. Chem. Soc. 1998, 120, 3411). To test this hypothesis, C-allylated derivatives of galactose, glucose, mannose and talose (10-15, FIG. 14), were synthesized to determine the influence of the nature and stereochemistry of the anomeric linkage on carbohydrate hydration and RI activity. Previously reported values for molar compressibility and hydration number for native monosaccharides are given for a mixture of anomers.

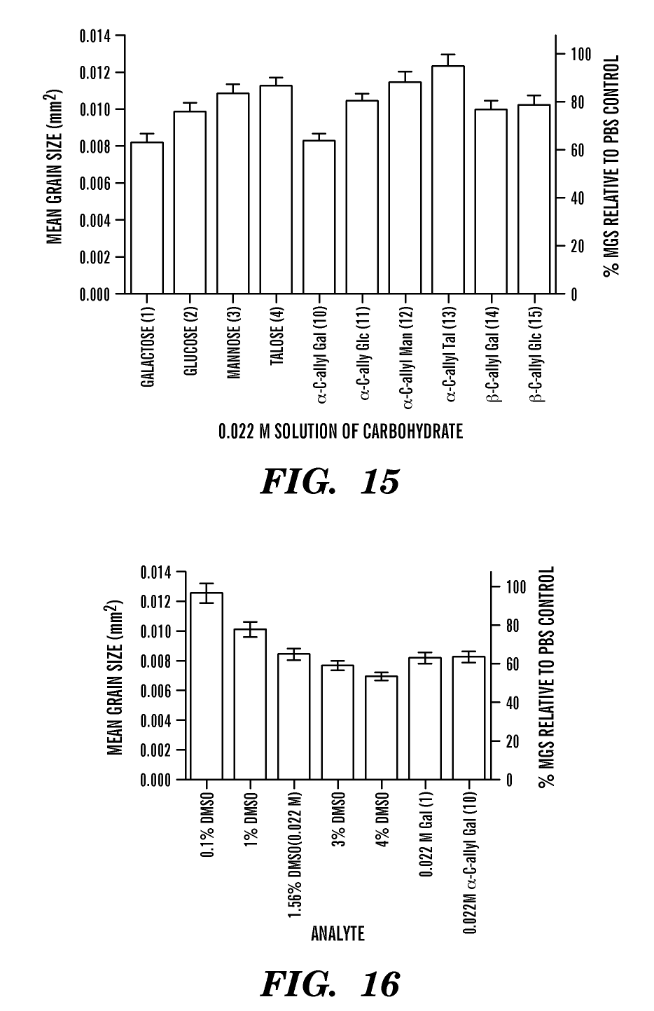

The RI activity of the C-linked analogues and their respective parent monosaccharides is shown in FIG. 15. Of all the α-C-allyl pyranoses (10-13), galactose derivative 10 possesses the most RI activity with mean grain sizes (MGS) statistically identical to native D-galactose. In fact, the RI activity trend of 10-13 is identical to their corresponding native monosaccharides 1-4, respectively. This is consistent with the hypothesis that the nature of the substituent at C1 has less influence on hydration than does the stereochemistry of the C2 and C4 hydroxyl groups. However, β-anomer 14 exhibits a marked decrease in RI activity relative to native O-linked galactose, 1, and α-C-linked derivative, 10, suggesting that anomeric configuration does play some role in RI activity and perhaps hydration. Comparatively, β-C-allyl glucose, 15, displayed the same amount of RI activity as its α-anomer, 11, with no statistically significant difference in MGS (Statistically significant differences are defined as p<0.05 based on a two-sample unequal variance Student's T-test). Further studies are required to ascertain the exact effect of anomeric configuration and its effect upon hydration and RI activity.

### Comparison to DMSO:

The effectiveness of galactose (1) and α-C-allyl galactose derivative 10 as a cryoprotectant for inhibiting recrystallization was assessed. This was accomplished using dimethylsulfoxide (DMSO) as a standard. DMSO is routinely used as an additive to cellular suspensions prior to freezing and storage at sub-zero temperatures (Heng, B. C.; Ye, C. P.; Liu, H.; Toh, W. S.; Rufiahah, A. J.; Yang, Z.; Bay, B. H.; Ge, Z.; Ouyang, H. W.; Lee, E. H.; Cao, T. J. Biomed. Sci. 2006, 13, 433-445; Ji, L.; de Pablo, J. J.; Palecek, S. P. Biotech. Bioengineering 2004, 88, 299-312). Despite the routine use of DMSO, it has many problems associated with its use, with the most significant being its cytotoxicity. Studies have shown that it elicits apoptosis in many different cell types (Heng, B. C.; Ye, C. P.; Liu, H.; Toh, W. S.; Rufiahah, A. J.; Yang, Z.; Bay, B. H.; Ge, Z.; Ouyang, H. W.; Lee, E. H.; Cao, T. J. Biomed. Sci. 2006, 13, 433-445; Ji, L.; de Pablo, J. J.; Palecek, S. P. Biotech. Bioengineering 2004, 88, 299-312). The generally accepted mechanism by which DMSO imparts cryopreservation is two-fold. Firstly it is thought to replace water in the cell membrane and thus prevent fractionation of the cell membrane during the thermatropic phase transition (Gurtovenko, A. A.; Anwar, J. J. Phys. Chem. 2007, 111, 10463-10460; Notman, R.; Noro, M.; O′Malley, B.; Anwar, J. J. Am. Chem. Soc. 2006, 128, 13982-13893). Secondly, molecular dynamics simulations have shown that individual DMSO molecules may cooperatively form extensive ion channels in the cell membrane and thus facilitate the transport of water into and out of the cell to relieve osmotic stress during the freezing event (Notman, R.; Noro, M.; O'Malley, B.; Anwar, J. J. Am. Chem. Soc. 2006, 128, 13982-13893). Regardless of the nature of the mechanism, the use of DMSO does result in improved viability of many cell types post freeze-thaw as compared to other protocols, and thus is used routinely as a standard in the industry (McGann, L. E.; Walterson, M. L Cryobiology 1987, 24, 11-16). Typically, DMSO concentrations employed in routine cryopreservation range between 1-20 mol %, but recent studies have shown that very little additional cryoprotection is conferred at concentrations above 5 mol % (Leseth, K.; Abrahamsen, J. F.; Bjorsvik, S,; Grottebo, K.; Bruserud, O. Cryotherapy, 2005, 4, 328-333). To the best of our knowledge, the ability of DMSO to function as an inhibitor of recrystallization has not yet been explored. Consequently, an initial concentration scan was performed using our RI assay. The results of this study are shown in FIG. 16.

As illustrated in FIG. 16, concentrations of 0.1 to 4% (v/v) DMSO were assessed. In practice, concentrations upwards of 6% (v/v) were very difficult to use in this assay and produced inconsistent results due to the large portions of unfrozen solution in the ice wafer. Consequently, only concentrations less than 6% were reliably assessed. A 0.1% DMSO solution failed to inhibit recrystallization and yielded ice crystals identical in size to the PBS control. In contrast, a 4% solution of DMSO produced a significant RI effect. The RI activity of a 4% DMSO solution is higher than a 0.022 M of galactose solution. As a benchmark for comparison between DMSO concentration and galactose concentration, a 0.022 M solution of galactose is as efficient at inhibiting recrystallization as a 3% (v/v) DMSO solution. These results suggest that galactose is useful as a potential cryoprotectant.

Proposed Recrystallization Inhibition Mechanism of Action with Ice:

As previously mentioned, it has been suggested that simple monosaccharides interact directly with the ice lattice based upon a complementarity of the pyranose hydroxyl groups with the ice lattice (Baruch, E.; Belostotskii, A. M.; Mastai, Y. J. Mol. Struct. 2008, 874, 170). As such, D-galactose contains hydroxyl groups which have the calculated optimal distance between them, approximately 4.2-4.5 Angstroms, and is the most potent inhibitor of recrystallization. Our most recent results suggest that the mechanism by which simple mono- and disaccharides inhibit recrystallization is more complex. For instance, a direct interaction between the carbohydrate and the ice lattice is unlikely based upon the fact that no thermal hysteresis or dynamic ice shaping is observed (Table 2). However, our studies show that carbohydrate stereochemistry does play some role in inhibiting the process of ice recrystallization, but the reason for this is unclear. We propose that hydration of the carbohydrate is a significant factor for inhibiting recrystallization of ice.

Our previous work tentatively linked isentropic molar compressibilities and carbohydrate stereochemistry to RI activity. The present experiments suggest that hydration numbers per molar volume constitutes a hydration index of the carbohydrate and this is a better predictor of RI activity (FIG. 13). We have demonstrated that carbohydrates with a large hydration index (such as galactose and melibiose) will have a greater effect on the ordering of bulk water surrounding the hydration shell of the carbohydrate and consequently are effective inhibitors of recrystallization. Heyden and coworkers have recently confirmed this long-range effect on bulk water using terahertz absorption measurements (Heyden, M.; Briindermann, E.; Heugen, U.; Niehues, G.; Leitner, D. M.; Havenith, M. J. Am. Chem. Soc. 2008, 130, 5773). This study demonstrates that solvated carbohydrates alter the long-range motion of water molecules by increasing the number of water-carbohydrate hydrogen bonds that can interact with water. Carbohydrates with lower hydration indices (i.e. talose and sucrose) will exert a minimal effect on the ordering of bulk water and would not be effective inhibitors of recrystallization. Given this premise, it seems likely that the carbohydrates are functioning at the QLL-bulk water interface and perturb the pre-ordering of bulk water, thus inhibiting transfer of a molecule of bulk water to the QLL. The overall result would be inhibition of recrystallization.

The mechanism of recrystallization has been studied extensively in the metallurgical literature within the context of inorganic composites (Pronk, P.; Infante Ferreira, C. A.; Witkamp, G. J. J. Crystal Growth 2005, 275, e1355; Huige, N. J. J.; Thijsenn, H. A. C. J. Crystal Growth 1972, 13/14, 483; Inada, T.; Modak, P. R.; Chem. Eng. Sci. 2006, 61, 3149; Kingery, W. D. J. Appl. Phys. 1959, 30, 301). In ice, there are two key issues that need to be considered. Firstly, a small amount of bulk water is present between adjacent ice crystals. Secondly, the interface of the ice lattice and bulk water is not an abrupt transition. Independent studies have proven that a layer of semi-ordered ice, or quasi-liquid layer (QLL), exists between the highly ordered ice lattice and bulk water (Karim, 0.; Haymet, A. D. J. Chem. Phys. Lett. 1987, 138, 531; Sadtchenko, V.; Ewing, G. E. J. Chem. Phys. 2002, 116, 4686). During the last several years, the nature and properties of this interfacial domain have been studied extensively and it has been implicated in the ability of antifreeze proteins to recognize ice (Madura, J. D.; Baran, K.; Wierbicki, A. J. Mol. Recognit. 2000, 13, 101). Consequently, a mechanism of action for inhibitors of ice recrystallization must account for the QLL.

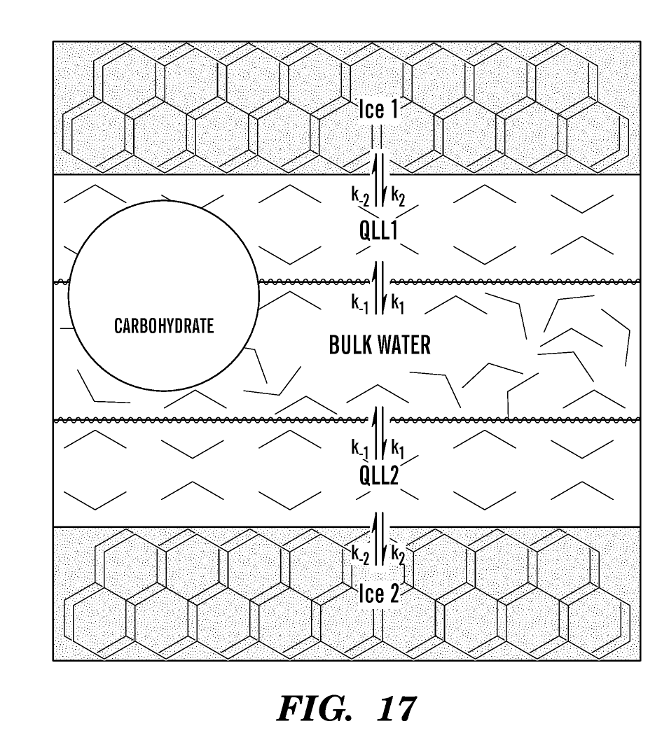

In the splat-cooling RI assay the wafer is frozen and contains very little unfrozen water. Furthermore, as solutes are excluded from the ice lattice the carbohydrate will be concentrated at the interface between two adjacent ice crystals and their QLLs. Consequently the localized concentration of carbohydrate in this region will be high. While the thickness of the bulk water region and the adjacent QLLs of neighbouring ice crystals are temperature dependant, the QLL is expected to be approximately 3-10 angstroms in thickness (Karim, O.; Haymet, A. D. J. Chem. Phys. Lett. 1987, 138, 531; Sadtchenko, V.; Ewing, G. E. J. Chem. Phys. 2002, 116, 4686). Taking all of this into account, a representation of a carbohydrate solvated in bulk water between two adjacent ice crystals and their QLLs is shown in FIG. 17.

The question of where the carbohydrate localizes with respect to the QLL is a difficult question to answer given the current inabilities to study the QLL. However, there are two possibilities. In the first, the carbohydrate is concentrated at the bulk water-QLL interface while in the second, the carbohydrate is actually incorporated into the QLL. The latter does not seem probable as we are not aware of any precedent for incorporation of a carbohydrate into the QLL. Furthermore, Uchida and co-workers have studied the RI activity of trehalose using TEM and proposed that trehalose functions at the bulk water-QLL interface (Uchida, T.; Nagayama, M.; Shibayama, T.; Gohara, K. J. Crys. Growth 2007, 299, 125).

A carbohydrate possessing a large hydration index positioned at this interfacial domain will disrupt the ordering of bulk water, leading to slightly increased energies associated with the transfer of bulk water to the QLL. Given that the thickness of the QLL is small and the entropy of the first hydration shell extends out to the third (Heyden, M.; Briindermann, E.; Heugen, U.; Niehues, G.; Leitner, D. M.; Havenith, M. J. Am. Chem. Soc. 2008, 130, 5773), this small energy difference associated with the transfer of bulk water to the QLL would be significant. Hence, the process of recrystallization will be slowed or inhibited. In instances where the carbohydrate contains a small hydration index and does not dramatically alter the solution structure of bulk water (such as talose), the transfer of a molecule of bulk water to the QLL will require comparatively little energy and the process of recrystallization will occur readily, resulting in larger crystals. In the case with disaccharides, larger absolute hydration numbers relative to smaller-sized monosaccharides do not necessarily translate to an increased inhibition of ice growth (FIG. 12). A disaccharide with a small hydration index such as sucrose has little effect upon recrystallization despite a larger volume than that of a monosaccharide such as galactose. The significance of this is that it shows not only does there need to be more water molecules in the hydration shell, but that they must also be highly concentrated around the solute, which may help to increase the entropy of surrounding bulk waters. Thus, more sparsely located water molecules in the hydration shell may have a lesser effect than more tightly packed water molecules. The net result is that despite the proportionally larger volume of a disaccharide relative to a monosaccharide, its ability to inhibit recrystallization may not be as good as a monosaccharide.

In addition, it is also feasible that compounds which are poor inhibitors of recrystallization may actually enhance the rate of transfer for a bulk water molecule from the QLL to the ice lattice. In other words, compounds which present a more ordered hydration shell on the “ice-facing side” may help in decreasing the entropy required for ice crystal formation.

### CONCLUSION

In summary, we have demonstrated that the hydration index of a carbohydrate is correlated to RI activity. The relationship between carbohydrate concentration and RI activity is non-colligative and hydration index modulates the ability of a sugar to function as a potent inhibitor of recrystallization. Amongst the monosaccharides examined in this study, galactose possesses the most RI activity while in the disaccharides, melibiose is the most potent. While C-linked derivatives of the pyranoses appear to parallel the RI activity of their O-linked native structures, the importance of anomeric stereochemistry in C-linked derivatives requires further investigation. A 0.022 M solution of C-linked galactose analogue (10) and D-galactose inhibit recrystallization as well as a 3% DMSO solution. Without wishing to be bound by any theory, but based upon the fact that the carbohydrates examined in this study did not possess any TH activity or dynamic ice shaping, we propose that they are inhibiting recrystallization at the bulk water-QLL interface by disrupting the pre-ordering of water.

### Experiment 3

### Conformation of C-Linked Antifreeze Glycoprotein Analogues and Its Effect on Antifreeze Activity

As shown above, a series of AFGP analogues have been prepared and shown to posses a high degree of ice recrystallization-inhibition activity (RI) and no thermal hysteresis. Analogues 2 and 3 (below) are two such inhibitors. The notion that these compounds may function as effective cryoprotectants is further exemplified by the fact that analogue 3 has been shown to exhibit little or no in vitro cytotoxicity, can penetrate cells and also inhibits cold-induced apoptotic cell death. The current experiment explores how decreasing the length of the amide-containing side chain influences antifreeze activity and how subtle changes in conformation of the side chain and carbohydrate affect the ability of these compounds to inhibit ice recrystallization.

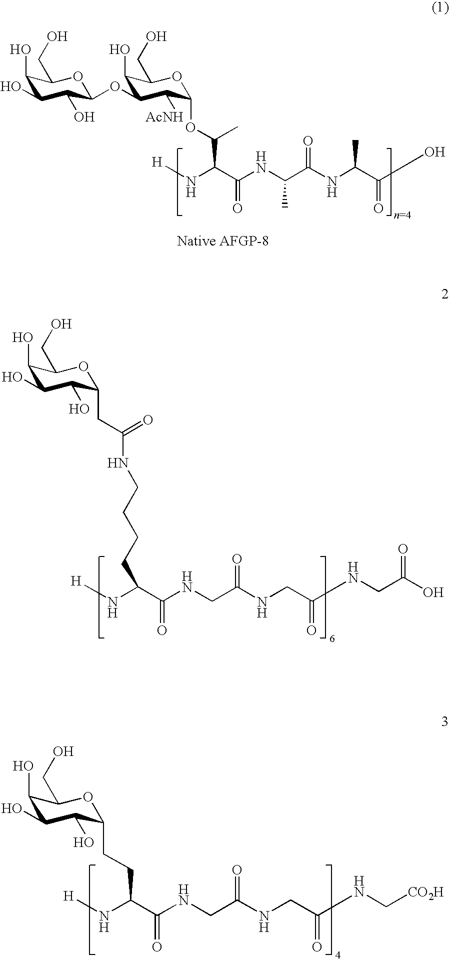

### Material and Methods:

### General Experimental:

All anhydrous reactions were performed in flame-dried glassware under a positive pressure of dry argon or nitrogen. Air or moisture-sensitive reagents and anhydrous solvents were transferred with oven-dried syringes or cannulae. All flash chromatography was performed with E. Merck silica gel 60 (230-400 mesh). Solution phase reactions were monitored using analytical thin layer chromatography (TLC) with 0.2 mm pre-coated silica gel aluminum plates 60 F254 (E. Merck). Components were visualized by illumination with a short-wavelength (254 nm) ultra-violet light and/or staining (ceric ammonium molybdate, potassium permanganate, or phosphomolybdate stain solution). All solvents used for anhydrous reactions were distilled. Tetrahydrofuran (THF) and diethyl ether were distilled from sodium/benzophenone under nitrogen. Dichloromethane, acetonitrile, triethylamine, and benzene were distilled from calcium hydride; diisopropylethylamine (DIPEA) was distilled from potassium hydroxide; methanol was distilled from calcium sulfate. N,N-dimethylformamide (DMF) was stored over activated 4 Å molecular sieves under argon. ¹H (400, or 500 MHz) and ¹³C NMR (100 or 125 MHz) spectra were recorded at ambient temperature on a Bruker Avance 400, Bruker Avance 500, or Varian Inova 500 spectrometer. Deuterated chloroform (CDCl₃), methanol (CD₃OD), or water (D₂O) were used as NMR solvents, unless otherwise stated. Chemical shifts are reported in ppm downfield from TMS and corrected using the solvent residual peak or TMS as an internal standard. Splitting patterns are designated as follows: s, singlet; d, doublet; t, triplet; q, quartet; m, multiplet and br, broad. Low resolution mass spectrometry (LRMS) was performed on a Micromass Quatro-LC Electrospray spectrometer with a pump rate of 20 μL/min using electrospray ionization (ESI) or a Voyager DE-Pro matrix-assisted desorption ionization-time of flight (MALDI-TOF), (Applied Biosystem, Foster City, Calif.) mass spectrometer operated in the reflectron/positive-ion mode with DHB in 20% Et₀H/H₂O as the MALDI matrix. High resolution mass spectrometry (HRMS) data was acquired on Applied Biosystems/Sciex QStar (Concord, ON). Samples in CH₂Cl₂/MeOH 1:1 were mixed with Agilent ES tuning mix for internal calibration, and infused into the mass spectrometer at 5 μL/min.

### Recrystallization-Inhibition (RI) Assay:

Samples were assayed for recrystallization-inhibition (RI) activity using the “splat cooling” method as described previously (Knight, C. A.; Hallet, J.; DeVries, A. L. Cryobiology 1988, 25, 55-60). A total of three images of the resulting ice wafer were photographed through a Leitz compound microscope equipped with an Olympus 20× (infinity corrected) objective with a Nikon CoolPix digital camera. Samples for analysis of ice crystal sizes were analyzed using the mean elliptical method. In this method, the ten largest ice crystals were chosen from the field of view (FOV) in each image. Selection of these crystals was arbitrary in that they were chosen after a visual inspection of the image. The two dimensional surface area of each of these ten crystals was then calculated via approximation of the crystal as an elliptical area. The major and minor elliptical axes were defined by the two largest orthogonal dimensions across the ice grain surface. The surface area of each ice grain was then calculated based on the formula: A=πab, in which A represented area; a and b represented the length of the major and minor elliptical axes. Totalling all individual measurements for each FOV produces a value for the average grain surface area referred to as the mean largest grain size (MLGS). Error was calculated using standard error of the mean (SEM). T-tests were performed to a 95% confidence level.

### Thermal Hysteresis Assay:

Nanoliter osmometry was performed using a Clifton nanoliter osmometer (Clifton Technical Physics, Hartford, N.Y.) as described by Chakrabartty and Hew (Chakrabartty, A.; Hew, C. L. Eur. J. Biochem. 1991, 202, 1057-1063). All measurements were made in double distilled water. Ice crystal morphology was observed through a Leitz compound microscope equipped with an Olympus 20× (infinity corrected) objective, Leitz Periplan 32× photo eyepiece and a Hitachi KP-M2U CCD camera connected to a Toshiba MV13K₁ TV/VCR system. Still images were captured directly using a Nikon CoolPix digital camera.

### Circular Dichroism:

CD spectra were obtained using a Jasco Model J-810 automatic recorder spectropolarimeter interfaced with a Dell computer. All measurements were performed at 22° C. in quartz cells of 0.1, 0.5 or 1.0 cm path lengths. Spectra were obtained with a 1.0 nm bandwidth time constant of 2 s, and a scan speed of 50 nm/min. Eight scans were added to improve the signal-to-noise ratio and baseline corrections were made against each sample. All the spectra were recorded between 190 nm and 300 nm and all the CD experiments were performed in doubly distilled H₂O at pH 7.4. Data obtained from CD spectroscopy was converted into molar ellipticity (deg cm² dmol⁻¹). Glycopeptide secondary structures were estimated using the deconvolution software CD Pro. The data from each spectrum was analyzed using three different deconvolution programs (SELCON3, CDSSTR, CONTINLL). Of the three programs, SELCON3 and CONTINLL gave the most consistent results. IBASIS 5 was used as the set of reference proteins containing 37 proteins with α-helix, β-structure, polyproline II and unordered conformations with optimal wavelength 185-240 nm (Sreerama, N.; Venyaminov, S. Y.; Woody, R. W. Anal. Biochem. 2000, 287, 243; Sreerama, N.; Woody, R. W. Anal. Biochem. 2000, 287, 252-260; Greenfield, N.J. Anal. Biochem. 1996, 35, 1-10).

### Variable Temperature NMR Studies:

Variable-temperature proton (500 MHz) spectra were recorded on a Varian Inova 500 spectrometer at temperatures ranging from 0° C. to 50° C. in 5-degree increments with a mixture of H₂O D₂O and D₂O (95:5) as solvent. An appropriate water suppression program was run and 2,2-dimethyl-2-silapentane-5-sulfonic acid (DSS) at a concentration of 20 ug/mL was used as a chemical shift internal standard (Hoffman, R. E.; Davies, D. B. Mag. Res. Chem. 1988, 26, 523-535).

### Molecular Dynamics Simulations:

Parameter Definitions: Solution conformations of the C-linked glycopeptide monomers and full length polymers were examined using MD simulations using AMBER 9 (Case, D. A.; Darden, T. E., Cheatham, III, C. L., Simmerling, J., Wang, R. E., Duke, R., Luo, K. M. M., D. A. Pearlman, M. Crowley, R. C. Walker, W. Zhang, B. Wang, S., Hayik, A. R., G. Seabra, K. F. Wong, F. Paesani, X. Wu, S. Brozell, V. Tsui, H., Gohlke, L. Y., C. Tan, J. Mongan, V. Hornak, G. Cui, P. Beroza, D. H. Mathews, C., Schafineister, W. S. R., and P. A. Kollman Eds.; University of California: San Francisco, 2006). Initial simulations modeled the truncated monomer of the full glycopeptides, consisting of an acetyl terminated glycine and an uncharged carboxyl glycine at the C-terminus. The acetyl and C-glycosylated amino acid types were defined as non-standard amino acids within the AMBER antechamber program. The GLYCAM Model developed by Woods and coworkers was used to assign partial charges to the carbohydrate (Woods, R. J.; Dwek, R. A.; Edge, C. J.; Fraser-Reid, B. J. Phys. Chem. 1995, 99, 3832). In our synthetic amino acids, the galactose is linked to the alkyl chain through a methylene unit instead of an oxygen at the C1 position; for this reason, the C1 was not constrained to its GLYCAM charge. RESP fitting (Bayl), C. I.; Cieplak, P.; Cornell, W.; Kollman, P. A. J. Phys. Chem. 1993, 97, 10269; Cieplak, P.; Cornell, W. D.; Bayl), C.; Kollman, P. A. J. Comput. Chem. 1995, 16, 1357) was then performed using electrostatic potentials generated from two HF/6-31G* minimized conformations where the (DM' angles of the amino acids were constrained to the α-helix (Φ=−60, Ψ=−40) and β-strand (Φ=−120, Ψ=140) conformations (Duan, Y.; Wu, C.; Chowdhury, S.; Lee, M. C.; Xiong, G.; Zhang, W.; Yang, R.; Cieplak, P.; Luo, R.; Lee, T.; Caldwell, J.; Wang, J.; Kollman, P. J. Comput. Chem. 2003, 24, 1999).

The monomer sequences were built using the tleap program in AMBER. The polypeptide was minimized using the generalized Born implicit solvent model with a 12 Å non-bonded cutoff, followed by annealing at 600 K for 100 ps, and then equilibration at 300 K for 100 ps. The equilibrated system was solvated in a TIP3P (Jorgensen, W. L.; Chandrasekhar, J.; Madura, J. D.; Impey, R. W.; Klein, M. L. J. Chem. Phys. 1983, 79, 926) water box with a 16 Å distance between the box edge and peptide, adding roughly 2500-3000 water molecules. The solvated peptide was then equilibrated with a 1 fs time step for 1 ns in the NPT ensemble, at 300 K and 1 bar, with a thermostat frequency of 5.0 ps⁻¹ and a barostat relaxation time of 2.0 ps. A 10 ns trajectory with frames recorded every 1 ps was generated for each tripeptide monomer starting with the equilibrated structures and utilizing the same simulation parameters as the equilibration step. The free energy profile of all galactose conformations was generated using Weighted Histogram Analysis Method (WHAM) (Kumar, S.; Rosenberg, J. M.; Bouzida, D.; Swendsen, R. H.; Kollman, P. A. J. Comput. Chem. 1995, 16, 1339) for ω6, which allowed us to quantify the energy differences between the chair and skew-boat conformations. Intramolecular hydrogen bonding analysis was performed using AMBER' s ptraj program.

Free energy profiles of rotations around the alkyl chain linker was generated by 324 MD simulations sampling the full 360 degrees of rotation for the angles restrained in 20° increments using a harmonic restraint with a spring constant of 10 kcal mol⁻¹ per degree. Each simulation was equilibrated for 80 ps with χ² and χ³ restrained at specific values. A 100 ps production run, sampling every 10 fs, was generated for all 324 combinations of angles adopted by χ² and χ³. The same procedure was then repeated for A4 with χ³ and χ⁴.

To model full-length glycopolymers, a modified procedure of the monomeric protocol was used. The critical glycopeptide and backbone torsion angles were restrained to the average torsional angles observed in the truncated monomer systems and then equilibrated them for 1 ns to generate a reasonable starting geometry. The peptides were then equilibrated without restraints for 1 ns before generating a 50 ns trajectory for all four systems.

### Results and Discussion:

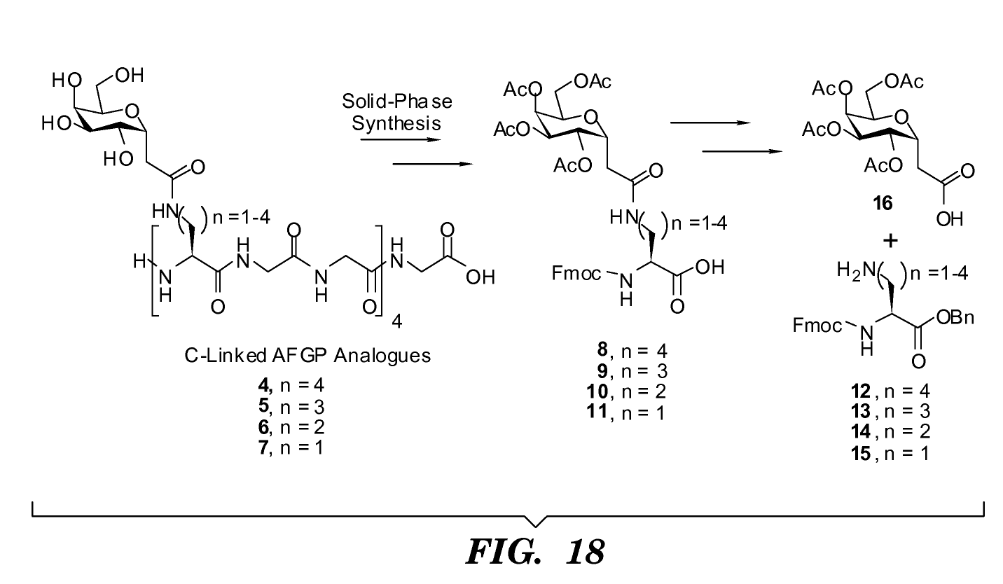

While assessment of antifreeze activity revealed an extremely small thermal hysteresis gap (0.02° C.), analogue 2 was a moderate inhibitor of ice recrystallization. This exciting result ultimately led to the design of C-linked AFGP analogue 3 which also possessed no TH activity but was an even more potent inhibitor of ice recrystallization. In fact, this analogue was equipotent to native AFGP 8 from Gagus ogac in its ability to inhibit ice recrystallization. Further investigations with analogues of 3 that possessed longer carbon side chains revealed that the ability to inhibit ice recrystallization was lost (Liu, S.; Ben, R. N. Org. Lett. 2005, 7, 2385). In an effort to understand this effect, several analogues of 2 containing shorter side chains were designed and synthesized (FIG. 18). It should be noted that compound 4 is a derivative of 2 but contains only four repeating tripeptide units instead of six. For the purpose of this study the synthesis of 4 was necessary given the well precedented relationship between glycopeptide length and antifreeze activity (Eniade, A.; Purushotham, M.; Ben, R. N.; Wang, J. B.; Horwath, K. Cell Biochem. Biophys. 2003, 38, 115; Eniade, A.; Murphy, A. V.; Landreau, G.; Ben, R. N. Bioconjugate Chem. 2001, 12, 817-823; Loudon, G. M.; Radhakrishna, A. S.; Almound, M. R.; Blodgett, J. K.; Boutin, R. H. J. Org. Chem., 1984, 49, 4272-4276; Zhang, L.; Kaufmann, G. S.; Pesti, J. A.; Yin, J. J. Org. Chem., 1997, 62, 6918-6920; Schmuck, C.; Geiger, L. Chem. Comm. 2005, 772-774), and thus permitted a direct comparison between analogues 5-7.

### Synthesis of C-linked Antifreeze Glycoprotein Analogues 4-7

The synthesis of these analogues was a convergent approach as previously reported where the C-linked carbohydrate moiety was coupled to the Fmoc-Lysine derivatives with side chains of appropriate length.

C-Linked AFGP analogues 4-7 were prepared from building blocks 8-11 (FIG. 18). Each glycoconjugate building block was prepared by coupling of the orthogonally protected building block (12-15) with C-linked pyranose derivative 16, the preparation of which has been previously reported (Fields, G. B.; Fields, C. G. J. Am. Chem. Soc. 1991, 113, 4202). Amino acid derivatives 12-13 were prepared from commercially available Fmoc-protected L-lysine and L-ornithine respectively but the key step to prepare 14 and 15 involved a Hoffman rearrangement performed on orthogonally protected L-glutamine and L-asparagine, respectively (Scheme 5).

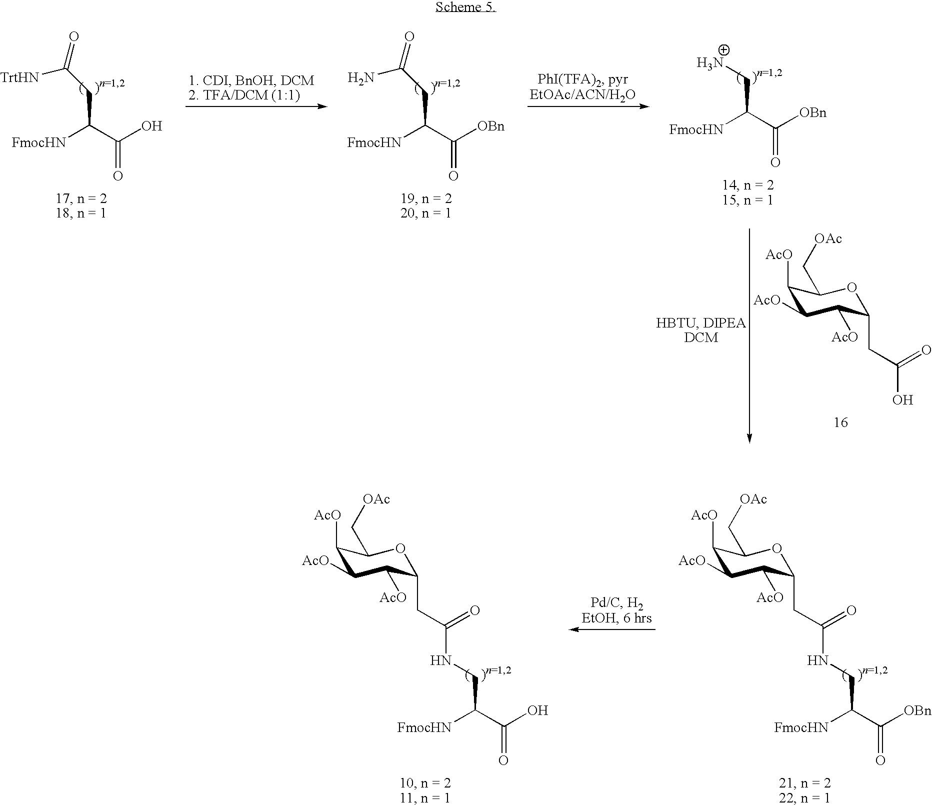

Commercially available trityl protected glutamine and asparagine (17 and 18) were reacted with 1,1-carbonyldiimidazole (CDI) in the presence of benzyl alcohol to yield the corresponding benzyl esters. Deprotection of the trityl protecting group using trifluoroacetic acid (TFA) furnished formamides 19 and 20 in quantitative yields. A modified Hoffmann reaction using bis(trifluoroacetoxyiodo)benzene (PIFA) (Loudon, G. M.; Radhakrishna, A. S.; Almound, M. R.; Blodgett, J. K.; Boutin, R. H. J. Org. Chem., 1984, 49, 4272-4276; Zhang, L.; Kaufmann, G. S.; Pesti, J. A.; Yin, J. J. Org. Chem., 1997, 62, 6918-6920; Schmuck, C.; Geiger, L. Chem. Comm. 2005, 772-774) produced the diaminobutanoic and diaminopropanoic amino acid derivatives 14 and 15 which were coupled to pyranose derivative 16 to furnish the respective glycoconjugates 21 and 22. Deprotection of the benzyl ester was accomplished using hydrogenolysis under atmospheric pressure to furnish building block 10 and 11. These building blocks were then utilized into the requisite C-linked AFGP analogues 6 and 7 using standard Fmoc-based automated solid phase synthesis as previously reported for analogues 4 and 5 (Eniade, A.; Purushotham, M.; Ben, R. N.; Wang, J. B.; Horwath, K. Cell Biochem. Biophys. 2003, 38, 115; Fields, G. B.; Fields, C. G. J. Am. Chem. Soc. 1991, 113, 4202).

### Evaluating Antifreeze Activity of C-Linked AFGP Analogues 4-7:

C-Linked AFGP analogues 4-7 were assessed for thermal hysteresis activity. All of these analogues failed to demonstrate any thermal hysteresis activity when tested with a Clifton nanoliter osmometer. Ornithine analogue 5 possessing a three carbon side chain between the polyamide backbone and amide bond exhibited weak dynamic ice shaping and produced single ice crystals with hexagonal morphology indicating the presence of a positive interaction with the ice surface (Knight, C. A., Cheng, C. C., DeVries, A. L. Biophys. J. 1991, 59, 409-418; Knight, C. A.; Driggers, E.; DeVries, A. L. Biophys. J. 1993, 64, 252-259; Pertaya, N.; Marshall, C. B.; DiPrinzio, C. L.; Wilen, L.; Thomson, E. S.; Wettlaufer, J. S.; Davies, P. L.; Braslaysky, I. Biophys. J. 2007, 92, 3663-3673). Analogues 6-7 did not show any dynamic ice shaping, suggesting that there is no interaction with the ice lattice or the quasi-liquid layer of ice. While this was an encouraging result, it was puzzling as a shortening of the side chain failed to produce analogues that interacted with the ice lattice in the same manner as three carbon analogue 5 did.

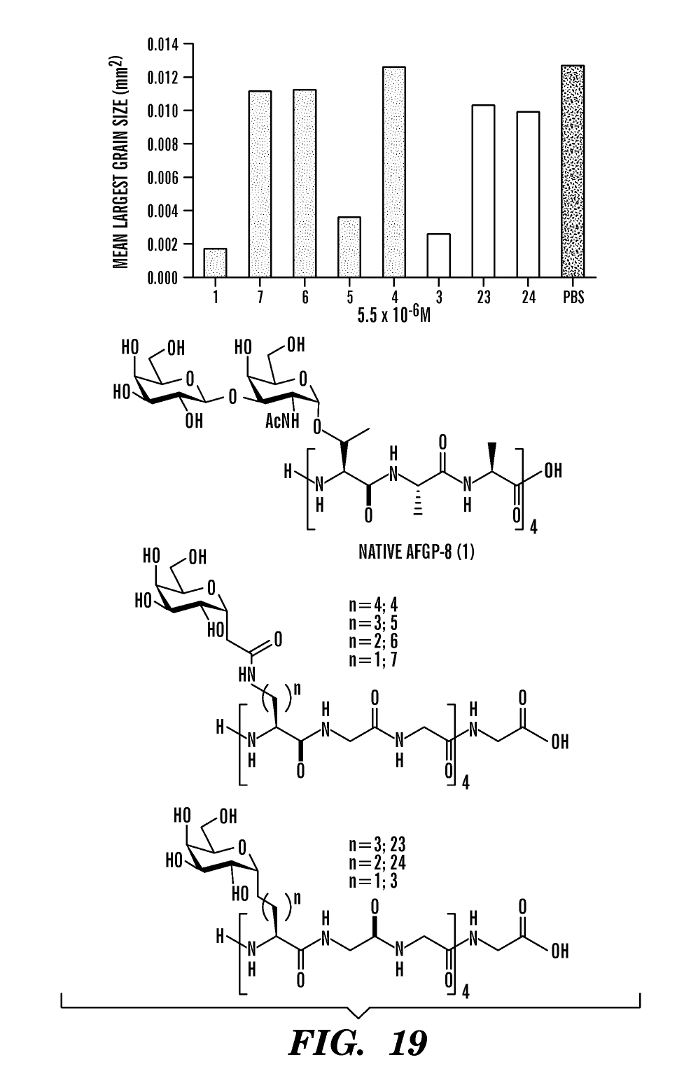

Glycoconjugates 4-7 were also assessed for the ability to inhibit ice recystallization (FIG. 19). In these measurements, native AFGP-8, 1, was used as a positive control for inhibition of ice recrystallization while phosphate buffered saline (PBS) represents a negative control. The Y-axis represents the mean largest ice crystal size and thus, larger values indicate large ice crystals and less inhibition of recrystallization. All samples were tested at 5.5 μM. In contrast to analogue 2, lysine analogue 4 possessed no RI activity when only four repeating tripeptide units were present in the polymer. This was not necessarily surprising as glycopolymer 4 contains only four repeating tripeptide units while 2 contains six. However, shortening the side chain of 4 by one carbon atom resulted in a dramatic increase in the ability of 5 to inhibit ice recrystallization. The trend is consistent with speculation that positioning the carbohydrate moiety closer to the polyamide backbone (akin to native AFGP 8 and C-linked AFGP analogue 3) imposes less conformational flexibility in the glycopeptide. This decreases the number of conformations accessible to the carbohydrate in solution making interactions with the ice-QLL more favorable resulting in increased RI activity. However, we were surprised that analogue 6 and 7, possessing two and one carbon atoms respectively, exhibited no RI activity despite a much closer proximity to the polypeptide backbone. We rationalized that this may be the result of a dramatic conformational change in the glycopeptide itself. As a result, the solution conformation of analogues 4-7 were studied using circular dichroism (CD), variable-temperature nuclear magnetic resonance (VT-NMR), and molecular dynamics (MD) simulations.

### Solution Conformations of C-Linked Analogues Via Circular Dichroism (CD) and NMR:

The solution conformation of native AFGP has been studied extensively using a variety of spectroscopic and computational (Tachibana, Y.; Fletcher, G. L.; Fujitani, N.; Tsuda, S.; Monde, K.; Nishimura, S. I. Angew. Chem. Int. Ed. 2004, 856-862) techniques. Previous spectroscopic studies have shown inconsistent results and the inability to obtain a suitable crystal for x-ray crystallographic analysis has prevented the unambiguous assignment of its true conformation. Vacuum ultraviolet circular dichroism (Bush, C. A.; Feeney, R. E.; Osuga, D. T.; Ralapati, S.; Yeh., Y. Int. J. Peptide Protein Res. 1981, 17, 125) and ¹H NMR (Bush, C. A.; Feeney, R. E. Int. J. Peptide Protein Res. 1986, 28, 386; Bush, C. A.; Ralapati, S.; Matson, G. M.; Yamasaki, R. B.; Osuga, D. T.; Yeh, Y.; Feeney, R. E. Arch. Biochem. Biophys. 1984, 232, 624) experiments have shown the dominant conformation to be a three-fold left-handed helix, whereas quasi-elastic light scattering (QELS) studies suggest an extended coil (Ahmed, A. I.; Feeney, R. E.; Osuga, D. T.; Yeh, Y. J. Biol. Chem. 1975, 250, 3344); other dynamic light scattering (DLS) (Bouvet, V. R.; Lorello, G. R.; Ben, R. N. Biomacromolecules 2006, 7, 565-571), CD (Bouvet, V. R.; Lorello, G. R.; Ben, R. N. Biomacromolecules 2006, 7, 565-571; Filira, R.; Biondi, L.; Scolaro, B.; Foffani, M. T.; Mammi, S.; Peggion, E.; Rocchi, R. Int. J. Biol. Macromol. 1990, 12, 41; Franks, F.; Morris, E. R. Biochem. Biophys. Acta. 1978, 540, 346; Raymond, J. A.; Radding, W.; DeVries, A. L. Biopolymers 1977, 16, 2575), ¹³C-NMR (Berman, E.; Allerhand, A.; DeVries, A. L. J. Biol. Chem. 1980, 255, 4407) studies suggest AFGP-8 predominantly exists as a random coil in solution.

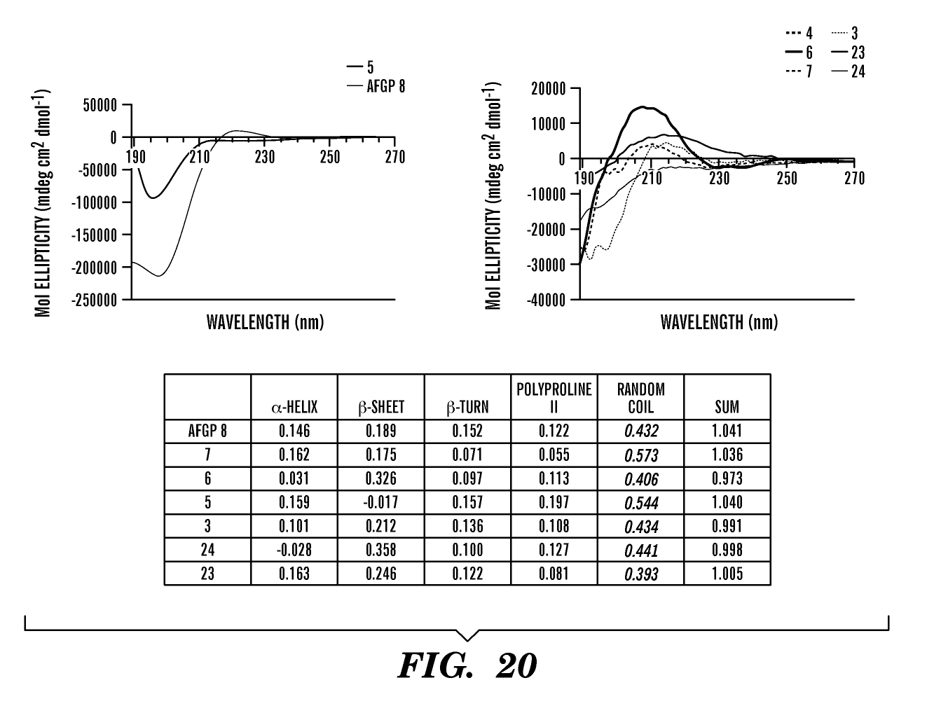

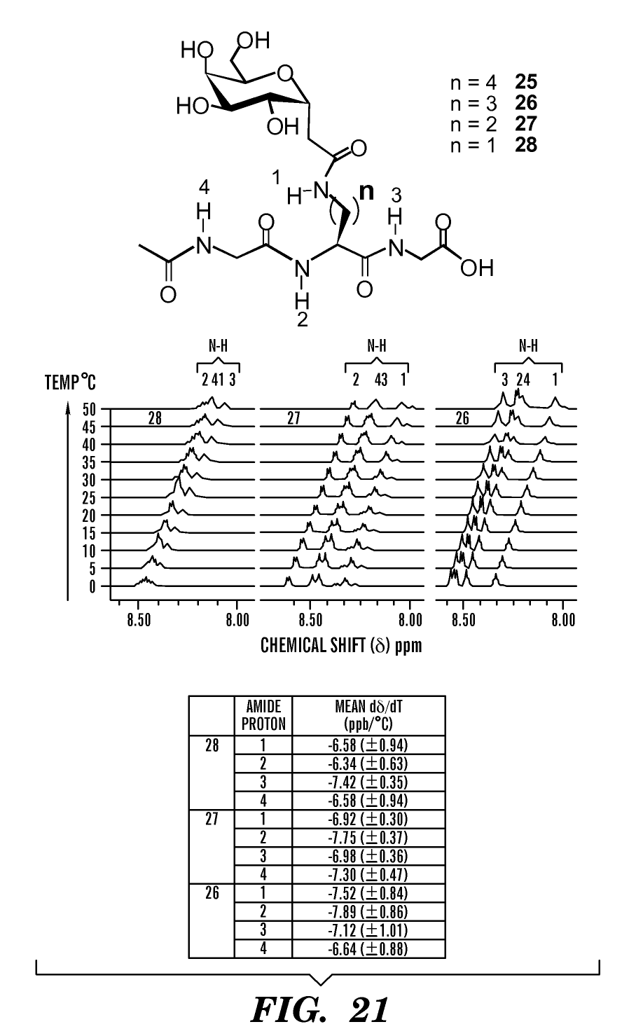

The CD spectra and deconvolution data of C-linked AFGP analogues 3-6, 23, 24 are shown in FIG. 20. These results suggest that all glycopolymers possess similar solution conformations on the timescale of CD and that the predominant solution conformation is that of random coil consistent with the most recent NMR studies of AFGP (Bouvet, V. R.; Lorello, G. R.; Ben, R. N. Biomacromolecules 2006, 7, 565-571; Filira, R.; Biondi, L.; Scolaro, B.; Foffani, M. T.; Mammi, S.; Peggion, E.; Rocchi, R. Int. J. Biol. Macromol. 1990, 12, 41; Franks, F.; Morris, E. R. Biochem. Biophys. Acta. 1978, 540, 346; Raymond, J. A.; Radding, W.; DeVries, A. L. Biopolymers 1977, 16, 2575; Berman, E.; Allerhand, A.; DeVries, A. L. J. Biol. Chem. 1980, 255, 4407). Thus we concluded that the long range conformation of these C-linked AFGP analogues does not appear to be very different than that of native AFGP 8, and the apparent loss in recrystallization inhibition activity observed in 6, 7, 23, 24 is not due to a dramatic change in solution conformation. However, while CD spectroscopy can often provide useful insight into the solution structure of proteins it cannot probe interactions between the carbohydrate moiety and the polypeptide backbone.

It has previously been proposed that the antifreeze activity of native AFGP-8 can be attributed to the orientation of the disaccharide relative to the backbone (Bush, C. A.; Feeney, R. E. Int. J. Peptide Protein Res. 1986, 28, 386; Bush, C. A.; Ralapati, S.; Matson, G. M.; Yamasaki, R. B.; Osuga, D. T.; Yeh, Y.; Feeney, R. E. Arch. Biochem. Biophys. 1984, 232, 624). Variable-temperature ¹H-NMR studies by Mimura have shown the existence of intramolecular hydrogen bonds between the amide proton of N-acetylgalactosamine (Ga1NHAc) and the carbonyl oxygen of threonine in monomeric model systems analogous to native AFGP-8 (Mimura, Y.; Yamamoto, Y.; Inoue, Y.; Chujo, R. Int. J. Biol. Macromol. 1992, 14, 242-248). In this technique, amide protons which are involved in stronger intramolecular hydrogen bonds exhibit a smaller change in chemical shift of the proton resonance signal as the temperature is increased (Ohnishi, M. and Urry, D. W. (1969) Biochem. Biophys. Res. Commun., 36, 194-202; Cierpicki, T.; Otlewski, J. J. Biomol. NMR 2001, 21, 249-261; Baxter, N. J.; Williamson, M. P. J. Biomolecular NMR. 1997, 9, 359-369). This temperature-dependant change in chemical shifts is known as the temperature coefficient and is represented by dδ/dT. To quantitatively correlate the strength of intramolecular hydrogen bonds with temperature coefficients, Cierpicki and Otlewski examined the temperature coefficients of 793 amide bonds from 14 proteins in H₂O/D₂O and by comparison with previously existing X-ray and NMR data, determined that values more positive than −3.2 ppb/° C. indicate that the proton is involved in a strong intramolecular hydrogen bond (Cierpicki, T.; Otlewski, J. J. Biomol. NMR 2001, 21, 249-261). Additional studies using this technique have reported values of −4.5 ppb/° C. (Baxter, N. J.; Williamson, M. P. J. Biomolecular NMR. 1997, 9, 359-369).

To investigate the possibility of intramolecular hydrogen bonding between the carbohydrate moiety and polypeptide backbone in 4-7 monomeric tripeptide units 26-28 were synthesized in a manner similar to 5-7. However, unlike glycopolymers 4-7, these truncated tripeptides were acetylated at the N-terminus. These monomers were then used to probe the solution conformation and/or intramolecular interactions between the carbohydrate moiety and the peptide backbone (Lane, A. N.; Hays, L. M.; Feeney, R. E.; Crowe, L. M.; Crowe, J. H. Protein Sci. 1998, 7, 1555-1563).

As a positive control, the full polymer of native AFGP-8 was also analyzed. Temperature coefficients of the galactosylacetamide N—H proton ranged from 4.62 to 8.4 ppb/° C. and correlated well with previous ¹H-NMR data for AFGPs (Mimura, Y.; Yamamoto, Y.; Inoue, Y.; Chujo, R. Int. J. Biol. Macromol. 1992, 14, 242-248; Lane, A. N.; Hays, L. M.; Feeney, R. E.; Crowe, L. M.; Crowe, J. H. Protein Sci. 1998, 7, 1555-1563). Taking this into account, the temperature coefficients of the acetamide N—H is 4.6 and 5.5 ppb/° C. and is consistent with glycoconjugate residues that are adjacent to only alanines suggesting a small degree of intramolecular hydrogen bonding involving the galactosylacetamide protons.

FIG. 21 shows the ¹H-NMR spectra of monomer model systems 26-28. Amide protons were assigned using 2-D COSY NMR (see supporting information for full ¹H and 2-D COSY NMR data). The temperature coefficients for all three monomers ranged from −6.34±0.63 ppb/° C. to −7.89±0.86 ppb/° C., which is significantly more negative than the threshold of 4.5 ppb/° C., suggesting no strong intramolecular hydrogen bonds persist between side chain and peptide backbone to enforce a local conformation. In addition, these results do not show any significant trend between temperature coefficients of each monomer and the IRI activity of the respective polymer. To further investigate the significance of variable side chain length and carbohydrate orientation on IRI activity, molecular dynamics simulations were employed to model the aqueous-phase conformational dynamics of selected analogues.

### Molecular Dynamics Simulations of C-Linked AFGP Analogue Monomers 25-28:

Monomeric tripeptides, 25-28, were modeled in an effort to identify key conformational differences between RI-active (5) and inactive (4, 6, 7) C-linked AFGP analogues.

The presence of intramolecular hydrogen bonding was examined using AMBER' s ptraj program and shows the absence of any significant persistent intramolecular hydrogen bonding between the amide protons and other hydrogen bonding acceptors (see supporting information for full analysis). This data correlates to the results of the variable temperature NMR studies previously described. The presence of brief intramolecular hydrogen bonds between the galactose and the backbone were found, but their occupancy are too low to account for any significant structural differences between the conformations adopted by 25-28. Corzana et al. (Corzana, F.; Busto, J. H.; Jimenez-Oses, G.; Asensio, J. L.; Jimenez-Barbero, J.; Peregrina, J. M.; Avenoza, A. J. Am. Chem. Soc. 2006, 128, 14640) recently reported that a direct intramolecular hydrogen bond between the N—H donor of a galactsosylacetamide and the threonine peptide backbone carbonyl acceptor was minimal in the solution conformation.

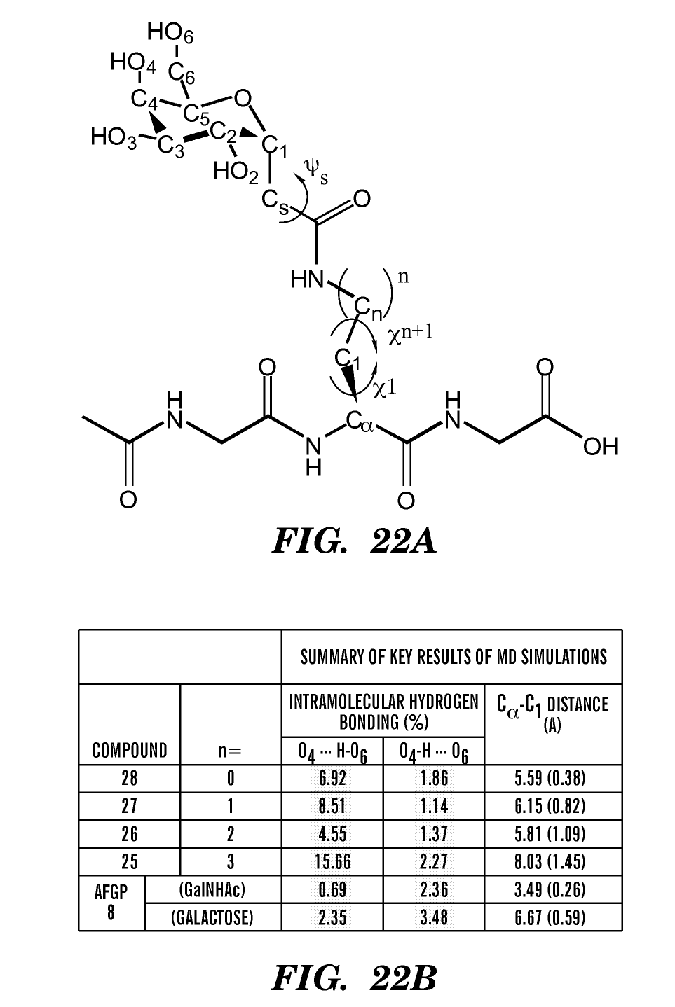

Interestingly, our study shows that the most prevalent intramolecular hydrogen bonding interactions are between the O4 and O6 hydroxyls of galactose, and that the amount of hydrogen bonding varies depending on the side-chain linker length (FIG. 22B). More specifically, stronger intramolecular hydrogen bonds exist between O4 and O6 for analogues 25, 27, 28 (all of which do not possess potent IRI activity) while lower percentages of hydrogen bonding between O4 and O6 exists for analogue 26 which is a potent inhibitor of ice recrystallization. A similar trend is also observed in both carbohydrate residues in native AFGP-8 system. This result implies that the ability of O4 and O6 with water molecules adjacent to the glycoprotein as opposed to each other may be an important requirement for ICR activity. The stereochemistry of the O4 hydroxyl has previously been shown to be an important factor in modulating the hydration environment of the carbohydrate (Galema, S. A.; Høiland, H. J. Phys. Chem. 1991, 95, 5321; Dashnau, J. L.; Sharp, K. A.; Vanderkooi, J. M. J. Phys. Chem. B 2005, 109, 24152) and we have subsequently herein verified that the degree of hydration is directly related to IRI activity.

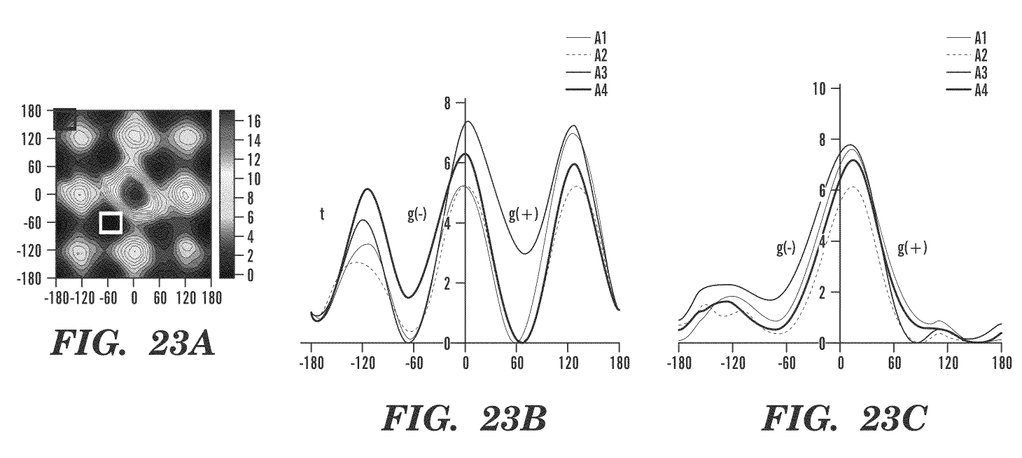

The Φ/Ψ torsional distributions for 25-28 were calculated, and suggest these four peptides adopt similar polypeptide backbone conformations (see supporting information). As such, we investigated how the relative orientation of the carbohydrate moiety relative to the backbone could modulate IRI activity. This was done by examining the torsion angles in the side chain (χ¹−χ^{N+1} and ψₛ) and the average distance from the C_{α} of the glycosylated amino acid residue to C1 of the carbohydrate, FIGS. 22B, 23.

The orientation of the side chain relative to the backbone depends on the rotation of the χ¹ torsion. To quantify this interaction, the free energy profile around the χ¹ torsional angle for 25-29 was calculated (FIG. 23B). The energy barrier of rotation at χ¹ is greater for 26 than for 25, 27 and 28, which signifies that the χ¹ torsional angle of 26 is much more restricted in its rotation.

Analysis of MD trajectories revealed the tendency of the χ² and χ³ angles in the alkyl chain of 26 to adopt a gauche (−), gauche (−), (g(−), g(−)), conformation, and the χ³ and χ⁴ angles in the longer alkyl chain of 25 to adopt the trans, trans (t,t) conformation (FIG. 23A). The g(−),g(−) torsion angles for χ² and χ³ of 26 causes the carbohydrate to be oriented almost parallel to the backbone, which forms a hydrophobic contact with the alkyl chain and peptide backbone and excludes all water molecules. This hydrophobic contact is not observed in 25, 27, 28, and the alkyl chain (and subsequently the carbohydrate) of these analogues extends in a trans-fashion into the solution throughout almost the full length of the 10 ns trajectory. Restraining the χ³ and χ⁴ torsional angles of 25 to (g(−), g(−)) allows this analogue to adopt a “folded back” structure with a hydrophobic contact between the carbohydrate moiety and side chain, at an energetic cost of 1-2 kcal mol⁻¹ increase in free energy. Increased conformational stability is formed from the hydrophobic contact in 26, and thus only the g(−) and t χ¹ rotamers are observed and explains the increased rotational energy barrier around χ¹ in 26. In contrast the side chains of 25, 27, and 28 are maximally extended into solution and have fewer interactions with the backbone which causes their χ¹ torsions to have a greater freedom of rotation.

The orientation of the carbohydrate relative to the backbone was determined based on the Ψₛ torsional angle in the C-glycosidic bond (FIG. 23C). Rotation of Ψₛ exposes different faces of the carbohydrate to the solvent. The hydrophobic contact formed in 26 favors a Ψₛ in the range of 60° to 180°. For Ψₛ in the range of −180° to 0°, the free energy of 26 is between 0.5-2.0 kcal mol⁻¹ higher than for 25, 27, and 28. Thus, as a result of the different side-chain conformations attributed to their varying lengths, the orientation of the carbohydrate relative to the peptide backbone in 26 is significantly different than the other three analogues. This may also explain the difference in intramolecular hydrogen bonding populations between the O4 and O6 of the galactose moiety.

From the various torsional angles data, the distance between the C1 of the carbohydrate and the Cα of the glycosylated amino acid were calculated (FIG. 22B). Monomers 26-28 have similar C_{α}-C1 distances of 5.59 Å, 6.15 Å, and 5.81 Årespectively; however, this distance is distinctly longer in 25 (8.03 Å). It was initially anticipated that this increase in the number of carbons in the side chain would result in a proportional increase in the Cα-C1 distance. However, our results show that analogue 26, a potent inhibitor of ice recrystallization, deviates significantly from this trend. Without wishing to be bound by any theory, we believe that this is due to the observed conformation of the side chain in which the side chain forms a hydrophobic contact with the peptide backbone and is ‘folded back’, compared to the other analogues which have more fully extended alkyl chains. In relation to the native AFGP-8 system, the distances between the galactosyl and GalNHAc C1 positions and the Cα are 6.67 and 3.49 Å respectively.

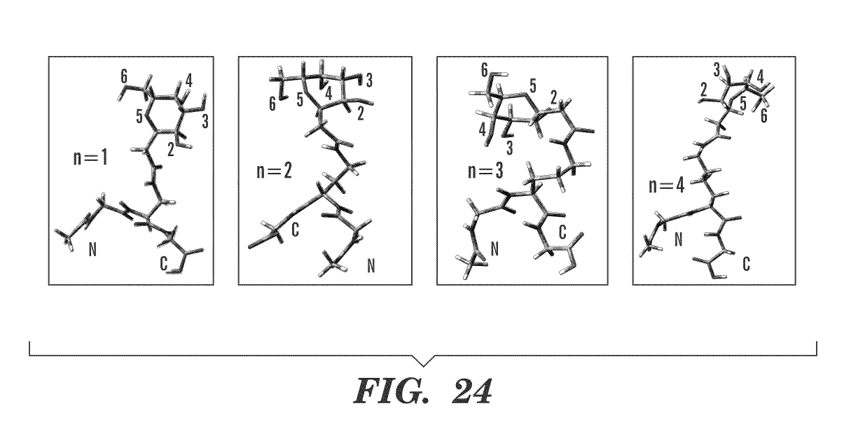

The overall lowest energy conformation of the various analogues takes into account the contributions from the various torsion angles, Cα-C1 distances, and inter-/intramolecular hydrophobic/hydrophilic interactions, and is shown in FIG. 24. From this figure, it is evident that there is a drastic conformational change between the RI-active (26) and the RI-inactive (25, 27, 28) analogues. The latter set of analogues have an extended alkyl side chain, which subsequently leads to a carbohydrate which has all of its faces exposed freely to the bulk solvent. Conversely, 26 contains a folded side chain which causes the hydrophilic face of the carbohydrate to be oriented towards the peptide backbone and away from the bulk solvent. We believe this is significant as this drastically alters the hydration shell of the overall glycoconjugate and hence peptide. Previous work has shown the important role hydration shells have on antifreeze activity.

### Full Systems

To confirm that our simulations of the model systems are relevant to the full polypeptides, we also generated MD simulations using the full synthetic AFGP analogues (4-7). Hydrogen bonding and torsional analysis of the full systems showed structures consistent with what was seen in 25-28. Calculation of the C_{α}-C1 distances for the full systems yielded the same results as the monomers; the C_{α}-C1 distances for 5-7 were all close to 6 Å, while this length was significantly larger for 8. The glycosylated side chains in the 5 system formed hydrophobic contacts with the backbone while the glycosylated residues for 4, 6, and 7 were extended.

### CONCLUSION

Described herein is the synthesis of several C-linked AFGP analogues bearing an amide-containing side chain of varying lengths between the carbohydrate moiety and polypeptide backbone. Analogue 5 possessing a three-carbon unit is a potent inhibitor of ice recrystallization while analogues 4, 6 and 7 possess no IRI activity. Analysis of solution conformation failed to indicate any significant conformation changes that might account for the observed difference activities. Consequently, the formation of intramolecular hydrogen bonds between the carbohydrate moiety and the polypeptide backbone or side chain was investigated but failed to verify the presence of strong intramolecular hydrogen bonds on the NMR timescale. However, detailed molecular dynamics simulations indicated that monomer 26 (a model for C-linked AFGP analogue 5) adopted a unique conformation in solution in which the carbohydrate moiety did not extend directly from the polypeptide backbone into the surrounding solution. In contrast, part of the side chain was folded back upon itself forming a hydrophobic “pocket” between the carbohydrate and the side chain. This appears to be a highly favorable interaction which restricts the available orientations of the carbohydrate. Without wishing to be bound by theory, this may be a favorable orientation for the carbohydrate moiety to interact with the quasi-liquid layer of the ice lattice resulting in potent IRI activity. The other C-linked AFGP analogues examined did not adopt such conformations in solution and fail to exhibit IRI activity. Without wishing to be bound by theory, it may be that formation of this hydrophobic pocket influences the hydration shell of the carbohydrate and this may also contribute to the observed IRI activity. This correlates well with other reports where the degree to which hydration shells influence hydration of carbohydrates in similar systems has been demonstrated.

Although this invention is described in detail with reference to exemplary embodiments, these embodiments are offered to illustrate but not to limit the invention. It is possible to make other embodiments that employ the principles of the invention and that fall within its scope as defined by the claims appended hereto. All scientific and patent publications cited herein are hereby incorporated in their entirety by reference.

1. An antifreeze glycoprotein comprising a polypeptide chain linked to at least one saccharide according to the following general formula: 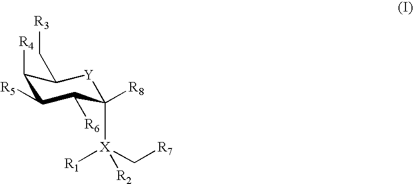
wherein:
X is nitrogen, sulfur, carbon or (CR¹R²)ₙS═O wherein n is 0, 1, 2, 3,
Y is carbon, nitrogen, sulfur or oxygen,
R¹ is hydrogen, methyl, fluorine, a carbohydrate, or a hydroxy chain,
R² is hydrogen, methyl, fluorine, a carbohydrate or a hydroxy chain,
R³ to R⁶ independently represent an alkyl chain, a hydroxy group, a dimethyl sulfoxy group, a carbohydrate or an ether,
R⁷ is ornithine, lysine, an alkyl chain, a pyridyl group or a heterocycle; and
R⁸ is hydrogen, fluorine a carbohydrate or a hydroxy or methoxy group.

2. The antifreeze glycoprotein according to claim 1, wherein R⁷ is ornithine or lysine and the ornithine or lysine is attached to the carbohydrate via an amide bond.

3. The antifreeze glycoprotein according to claim 1, wherein the carbohydrate is selected from galactose, glucose, fructose, L-fucose, lactose, melibiose and lactose(NAc).

4. The antifreeze glycoprotein according to claim 1, wherein the heterocycle is selected from: 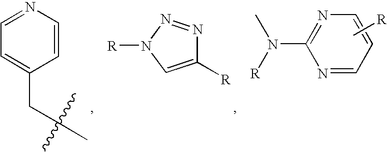
wherein R represents hydrogen, an alkyl chain or an aromatic group.

5. The antifreeze glycoprotein according to claim 1, wherein X is carbon.

6. The antifreeze glycoprotein according to claim 1, wherein Y is oxygen.

7. The antifreeze glycoprotein according to claim 1, wherein R³ to R⁶ each represent a hydroxy group.

8. The antifreeze glycoprotein according to claim 1, wherein R³, R⁴ and R⁶ each represent a hydroxy group, and R⁵ represents a carbohydrate.

9. The antifreeze glycoprotein according to claim 8, wherein the carbohydrate of R⁵ comprises a saccharide selected from galactose, glucose, fructose, L-fucose, lactose, melibiose and lactose(NAc) and is linked to the sugar moiety of formula (I) via an alpha- or beta-linkage.

10. The antifreeze glycoprotein according to claim 1, wherein the main saccharide unit is a galactose, glucose, fructose, L-fucose, lactose, melibiose, or lactose(NAc).

11. The antifreeze glycoprotein according to claim 1, wherein the main saccharide unit is a dimerized monosaccharide according to formulae (IV): 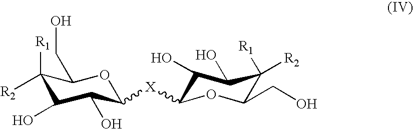
wherein R¹ and R² represent hydrogen, a halogen or a hydroxyl group, and X is CH₂, CF₂, oxygen or sulfur, and wherein the stereochemical nature of the dimer linkage is either alpha or beta.

12. The antifreeze glycoprotein according to claim 1, wherein the main saccharide unit is represented according to the following formulae (II) or (III): 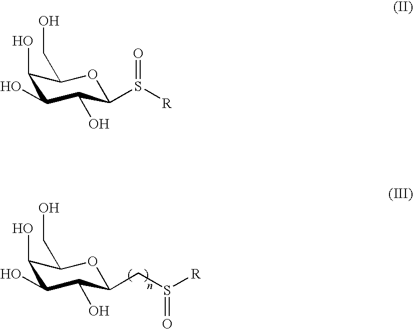
wherein R represents an alkyl chain or an aryl group.

13. The antifreeze glycoprotein according to claim 12, wherein R represents a substituted or unsubstituted phenyl group, wherein the substituents are selected from alkyl, ester, amide or carboxylic acid substituents.

14. The antifreeze glycoprotein according to claim 1, wherein the saccharide forms part of a glycoconjugate within the polypeptide chain.

15. The antifreeze glycoprotein according to claim 14, wherein the polypeptide chain comprises a repeating polypeptide unit.

16. The antifreeze glycoprotein according to claim 15, wherein R⁷ is ornithine and the repeating polypeptide unit comprises (ornithine-aa¹-aa²)ₙ, wherein aa¹ and aa² each represent an amino acid selected from alanine and glycine, and n is 3 or 4.

17. The antifreeze glycoprotein according to claim 16, wherein the repeating polypeptide unit comprises a tripeptide repeat of (ornithine-Gly-Gly).

18. The antifreeze glycoprotein according to claim 16, wherein the antifreeze glycoprotein comprises four (ornithine-Gly-Gly) tripeptide repeating units selected from any one of the following formulae (V), (VI) and (VII):

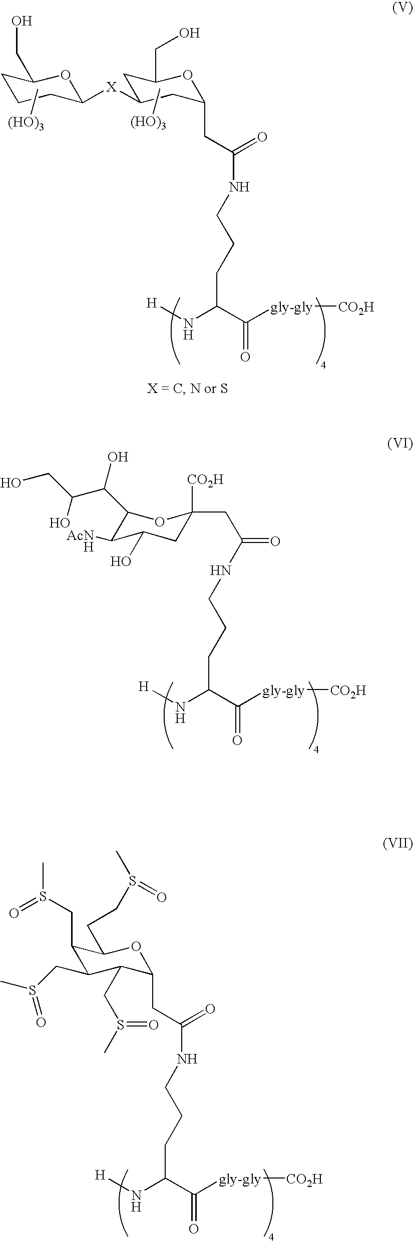

19. A saccharide unit for an antifreeze glycoprotein analog according to the following general formula: 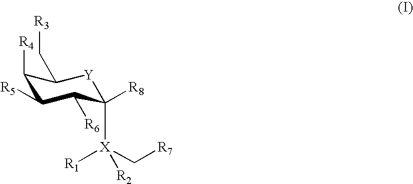
wherein:
X is nitrogen, sulfur, carbon or (CR¹R²)ₙS═O wherein n is 0, 1, 2, 3,
Y is carbon, nitrogen, sulfur or oxygen,
R¹ is hydrogen, methyl, fluorine, a carbohydrate, or a hydroxy chain,
R² is hydrogen, methyl, fluorine, a carbohydrate or a hydroxy chain,
R³ to R⁶ independently represent an alkyl chain, a hydroxy group, a dimethyl sulfoxy group, a carbohydrate or an ether,
R⁷ is ornithine, lysine, an alkyl chain, a pyridyl group or a heterocycle; and
R⁸ is hydrogen, fluorine a carbohydrate or a hydroxy or methoxy group.

20. A method of inhibiting recrystallization comprising adding an antifreeze glycoprotein analogue according to claim 1 to a material in need thereof in an amount sufficient to inhibit recrystallization thereof.

21. The method according to claim 20, wherein the antifreeze glycoprotein analogue is added to a material as a cryoprotectant for tissue preservation and/or transplantation, for improving the texture of processed frozen food, for frostbite protection, for crop protection, or is added to a composition for land vehicle antifreeze and aircraft de-icing.
